(19) United States
(12) Patent Application Publication
(10) Pub. No.: US 2022/0193085 A1
(43) Pub. Date: Jun. 23, 2022

(54) METHODS OF THROMBOPROPHYLAXIS

(71) Applicants: JANSSEN PHARMACEUTICALS, INC., Titusville, NJ (US); BAYER PHARMA ATKIENGESELLSCHAFT, Berlin (DE)

(72) Inventors: CHRISTOPHER NESSEL, Doylestown, PA (US); Stefan Willmann, Dusseldorf (DE); Dagmar Kubitza, Ratingen (DE); Roger M. Mills, Chagrin Falls, OH (US); Catherine Vanden Boom, Perkasie, PA (US); Elliot Barnathan, Havertown, PA (US); Liza Miriam Pina, Fleminnton, NJ (US)

(73) Assignees: JANSSEN PHARMACEUTICALS, INC., Titusville, NJ (US); BAYER PHARMA ATKIENGESELLSCHAFT, Berlin (DE)

(21) Appl. No.: 17/553,340

(22) Filed: Dec. 16, 2021

Related U.S. Application Data
(60) Provisional application No. 63/126,908, filed on Dec. 17, 2020.
(60) Provisional application No. 63/212,170, filed on Jun. 18, 2021.

(51) Int. Cl. A61K 31/5377; A61K 9/00; A61P 7/02

(52) U.S. Cl. CPC A61K 31/5377; A61P 7/02; A61K 9/0053

(57) ABSTRACT

Methods of thromboprophylaxis using rivaroxaban is provided for children with congenital heart disease post-Fontan procedure.

### CROSS-REFERENCE TO RELATED APPLICATIONS

This application claims priority to U.S. Provisional Patent Application No. 63/212,170, filed on Jun. 18, 2021, and U.S. Provisional Patent Application No. 63/126,908, filed Dec. 17, 2020. Each disclosure is incorporated herein by reference in its entirety.

### FIELD OF INVENTION

The present invention relates to methods of thromboprophylaxis in pediatric patients 2 years and older with congenital heart disease after the Fontan procedure.

### BACKGROUND OF INVENTION

Thrombotic and/or thromboembolic events remain an important concern and potential complication for patients following the Fontan procedure, and particularly in pediatric patients with congenital heart disease (CHD). Manlhiot C, et al. Thrombotic complications and thromboprophylaxis across all three stages of single ventricle heart palliation. J Pediatr. 161: 513-9 (2012). Based on estimates in multiple studies, the occurrence of thromboembolic events post-Fontan procedure ranges from 17% to 33%, with a reported mortality of 25% to 38%. Balling G, et al. Intracardiac thrombus formation after the Fontan operation. The Journal of Thoracic and Cardiovascular Surgery. 119: 745-52 (2000); Firdouse M, et al. Thrombosis and thromboembolic complications in fontan patients: a literature review. Clin Appl Thromb Hemost. 20: 484-92 (2014); Fyfe D A, et al. Transesophageal echocardiography detects thrombus formation not identified by transthoracic echocardiography after the fontan operation. Journal of the American College of Cardiology. 18: 1733-7 (1991).

Neither the literature nor routine clinical practice for the optimal type of antithrombotic therapy or the duration of therapy for thromboprophylaxis after Fontan surgery has provided a suitable approach to thromboprophylaxis for the patient subgroup. Giglia™, et al. American Heart Association Congenital Heart Defects Committee of the Council on Cardiovascular Disease in the Young CoC, Stroke Nursing CoE, Prevention, Stroke C. Prevention and treatment of thrombosis in pediatric and congenital heart disease: a scientific statement from the American Heart Association. Circulation. 128: 2622-703 (2013); Monagle P, et al. Antithrombotic therapy in neonates and children: Antithrombotic Therapy and Prevention of Thrombosis, 9th ed: American College of Chest Physicians Evidence-Based Clinical Practice Guidelines. Chest. 141: e737S-e801S (2012).

Alternative thromboprophylaxis strategies are needed in the post-Fontan population considering the high prevalence of thrombotic events, difficulties achieving consistent protection with current anticoagulant therapy, and the considerable residual risk that remains in children treated with warfarin or acetylsalicylic acid (ASA).

Rivaroxaban, chemical name 5-chloro-N-({(5S)-2-oxo-3-[4-(3-oxo-4-morpholinyl)phenyl]-1,3,oxazolidin-5-yl}methyl)-2-thiophenecarboxamide, is a medication used for the treatment and prevention of thrombotic and thromboembolic disorders. Rivaroxaban has been evaluated for the treatment of acute venous thromboembolism (VTE) in children aged 0-18 years in the EINSTEIN-Jr program, Lensing AWA, et al. Rivaroxaban versus standard anticoagulation for acute venous thromboembolism in childhood. Design of the EINSTEIN-Jr phase III study. Thromb J. 16: 34 (2018); Young G, et al. Rivaroxaban for Treatment of Pediatric Venous Thromboembolism. an Einstein-Jr Phase 3 Dose-Exposure-Response Evaluation. Blood. 134: 164 (2019); however, there remains a need for appropriate thromboprophylaxis in children aged 2 to 18 years with CHD post-Fontan procedure. Pina L M, et al. Rivaroxaban, a direct Factor Xa inhibitor, versus acetylsalicylic acid as thromboprophylaxis in children post-Fontan procedure: Rationale and design of a prospective, randomized trial (the UNIVERSE study). Am Heart J. 213: 97-104 (2019).

This invention addresses the need in the art for thromboprophylaxis in post-Fontan pediatric patients.

### SUMMARY

The invention concerns the use of rivaroxaban (5-chloro-N-({(5S)-2-oxo-3-[4-(3-oxo-4-morpholinyl)phenyl]-1,3,oxazolidin-5-yl}methyl)-2-thiophenecarboxamide) for thromboprophylaxis against thrombotic events in children, e.g., aged 2 years up to 18 years of age, with congenital heart disease after the Fontan procedure.

In one aspect, the invention relates to methods of prophylactic treatment against thrombotic/thromboembolic events (thromboprophylaxis) in a patient post-Fontan procedure, the method comprising administering to said patient rivaroxaban in an amount clinically proven safe and/or clinically proven effective for thromboprophylaxis in pediatric patients 2 years and older with congenital heart disease after the Fontan procedure.

In another aspect, the invention relates to methods of thromboprophylaxis in a patient post-Fontan procedure, the method comprising administering to said patient rivaroxaban in an amount clinically proven safe and/or clinically proven effective, wherein after the start of said treatment the patient is a responder to said treatment as measured by the absence of the incidence of any thrombotic event in the patient for a period of 6 months or more, preferably 12 months or more, from first administration.

In another aspect, the invention relates to methods of thromboprophylaxis against thrombotic/thromboembolic events associated with an extracardiac conduit in a patient post-Fontan procedure, the method comprising administering to said patient having an extracardiac conduit rivaroxaban in an amount clinically proven safe and/or clinically proven effective for thromboprophylaxis in pediatric patients 2 years and older with congenital heart disease after the Fontan procedure.

In another aspect, the invention relates to methods of thromboprophylaxis in a pediatric patient of 2 years up to 18 years after a Fontan procedure, the method comprising orally administering to said patient once daily or twice daily rivaroxaban in an amount clinically proven safe and clinically proven effective for thromboprophylaxis, wherein the administering results in a steady-state exposure of rivaroxaban at a geometric mean exposure AUC_{ss,24h} of 70% to 143% of AUC_{ss,24h} 1494 μg*h/L.

In another aspect, the invention relates to a method of thromboprophylaxis in a pediatric patient at least two year old after a Fontan procedure, the method comprising orally administering to said patient once daily or twice daily rivaroxaban in an amount clinically proven safe and/or clinically proven effective for thromboprophylaxis, wherein the administering results in a steady-state exposure of rivaroxaban at a geometric mean exposure AUC_{ss,24h} that is within the 2.5th to 97.5th percentile exposure range of the adult reference of AUC_{ss,24h} 1494 μg*h/L.

In another aspect, the invention relates to a method of thromboprophylaxis in a pediatric patient at least two year old after a Fontan procedure, the method comprising orally administering to said patient once daily or twice daily rivaroxaban in an amount clinically proven safe and/or clinically proven effective for thromboprophylaxis, wherein the administering results in a steady-state exposure of rivaroxaban at a geometric mean exposure AUC_{ss,24h} that is within the range of 1317 to 1576 μg*h/L.

### BRIEF DESCRIPTION OF DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a diagram of the study flow. Completed Treatment=Received treatment for 12 months.

FIG. 2 shows a graph of the results of primary efficacy in Part B of study, the randomized part of the study.

FIG. 3 shows a graph of the results of the safety outcomes in Part B of study.

FIG. 4 shows body weight-adjusted dosing table proposed for UNIVERSE (daily dose, administered BID), comparison of rivaroxaban exposure (AUC_{24h, steady state}) and steady-state plasma concentration-time profile of rivaroxaban predicted for post-Fontan patients aged 2-8 years in comparison to healthy pediatric and/or adult reference population for the proposed dosing regimen.

FIG. 5 shows a graph demonstrating prothrombin time (PT) versus plasma rivaroxaban concentrations: comparison between the UNVERSE study and the adult reference in studies ODIXa-HIP2 and ODIXa-KNEE.

FIG. 6 shows a graph demonstrating activated partial thromboplastin time (aPTT) versus rivaroxaban concentrations: comparison between the UNVERSE study and the adult reference studies ODIXa-HIP2 and ODIXa-KNEE.

FIG. 7 shows a graph demonstrating anti-factor Xa activity vs. plasma rivaroxaban concentrations in the UNIVERSE study. The solid line represents the median of model prediction; the dashed lines represent the 95% prediction interval. Regression line: y=−3.14+0.885*x; R-squared=0.901.

FIG. 8 shows exposure-thrombosis event relationship of rivaroxaban in the UNIVERSE study. AUC_{24h,ss}=area under the plasma concentration-time curve to 24 hours at steady-state; C_{max,ss}=post-dose maximum concentration at steady-state; C_{trough,ss}=predose concentrations at steady-state; VTE=venous thromboembolism. Note: Individual values are provided where n=1. Note: The solid line in the box is the median. The boundaries of the box represent the 25^{th} and 75^{th} percentiles. The whiskers are the nearest values within 1.5 times the inter-quartile range below and above the 25th and 75th percentile respectively.

FIG. 9 shows exposure-bleeding events relationship of rivaroxaban in the UNIVERSE Study. AUC_{24h,ss}=area under the plasma concentration-time curve to 24 hours at steady-state; C_{max,ss}=post-dose maximum concentration at steady-state; CRNM=clinically relevant non-major bleeding; C_{trough,ss}=predose concentrations at steady-state. Note: all bleeding includes the 3 bleeding categories: major, trivial, and CRNM. Note: The solid line in the box is the median. The boundaries of the box represent the 25^{th} and 75^{th} percentiles. The whiskers are the nearest values within 1.5 times the inter-quartile range below and above the 25th and 75th percentile respectively.

FIG. 10 shows the outline of the database and concept for the bridging approach for dosing to post-Fontan patients aged to 18 years. VTE=venous thromboembolism; T: treatment indication (for patients who already have a VTE); P: prevention indication (prophylactic use to avoid VTE).

FIG. 11 shows PBPK model-predicted body weight dependence of AUC(0-24)ₛₛ in pediatric post-Fontan patients aged 2 to 18 years. On the right-hand side adult and pediatric references are shown. The solid line is the simulated post-Fontan pediatric subjects geometric mean; the dashed line is the simulated post-Fontan pediatric subjects with severe hepatic impairment 95th percentile; the grey area is post-Fontan pediatric subjects 5^{th} to 95^{th} percentile. The reference studies are: (A) healthy adults (10 mg OD, N=263); (B) adult VTE-P patients (10 mg OD, N=140); (C) Medical ill (10 mg OD, N=35); (D) Chronic stable severe CHF (10 mg OD, N=12); (E) Acute decomp. CHF (10 mg OD, N=3); (F) EINSTEIN-JR (Study 14372, patients with BW 30 kg: 15 mg OD (BW from 30-<50 kg) and 20 mg OD (BW 50 kg), N=201. Reference data are displayed as box whisker plot (B,F), which indicates the percentiles 5, 25, 50, 75, and 95 or geometric mean+range (A,C,D,E).

FIG. 12 shows comparison of rivaroxaban AUC(0-24)ₛₛ predicted by the EINSTEIN-JR popPK model and PBPK model for pediatric post-Fontan patients with body weights between 30 and <50 kg receiving 7.5 mg once daily and body weights ≥50 kg receiving 10 mg once daily. For comparison, exposure data obtained after 10 mg in healthy adults, medically ill patients, orthopedic surgery patients, and chronic and acute CHF patients, as well as exposure data obtained in pediatric VTE patients with body weight 30 kg receiving 15 mg (30 to <50 kg) or 20 mg once daily (OD) (≥50 kg) are also shown. For simulated data and adult VTE-P patients and EINSTEIN-JR studies: Whiskers (black bars) represent the range between the 5^{th} and 95^{th} parameter value. The box represents the range between lower and upper quartile. The black horizontal line represents the median. For healthy adults, medically and chronic and acute CHF patients: Whiskers (black bars) represents the range between the minimum and maximum value; the black horizontal line represents the median. The shaded area represents the range between the 5th and 95th parameter value in the adult population (VTE-P study) the solid line within the range represents the median in the adult population.

### DETAILED DESCRIPTION OF THE INVENTION

The present invention relates to the discovery that rivaroxaban may be used in a method of thromboprophylaxis in a patient post-Fontan procedure. The methods may comprise administering rivaroxaban in an amount clinically proven safe and/or clinically proven effective for thromboprophylaxis in pediatric patients 2 years and older with congenital heart disease after the Fontan procedure.

Rivaroxaban may be prepared as described in WO 01/47919 and is commercially available. Formulations of rivaroxaban are known in the art and include the formulations disclosed in U.S. Pat. No. 7,157,456 to Straub et al., issued Jan. 2, 2007 and in U.S. Pat. No. 9,402,851 to Benke, issued Aug. 2, 2016.

As will be understood, terms such as “patient,” “pediatric patient,” “child,” or “subject in need” are used in reference to such individuals from ages 2 to 18 years of age. In certain embodiments, the patient, pediatric patient, child, or subject in need are individuals from ages 2 to 8 years of age.

As will be appreciated herein, thrombotic events of concern in patients post-Fontan procedure may include, in part, superior vena cava occlusion, inferior vena cava occlusion, arterial thrombosis, venous thrombosis, thromboembolism, pulmonary embolism, and other complications known to be associated in pediatric patients with CHD in post-Fontan procedure scenarios. In certain embodiments, the present methods provide thromboprophylaxis against all such thrombotic/thromboembolic events. As will be understood herein, the Fontan procedure involves open-heart surgery wherein the inferior vena cava (IVC) is disconnected from the heart and attached to the pulmonary artery, often using an extracardiac conduit to connect the IVC to the pulmonary artery. In certain embodiments, the methods of the invention provide thromboprophylaxis against conduit-related thrombosis, for example, against the formation of thromboses within or associated with the extracardiac conduit. In certain embodiments of the present methods, the patient is a responder to said method of thromboprophylaxis as measured by the absence of the incidence of any thrombotic/thromboembolic event associated with the extracardiac conduit in the patient for a period of 6 months or more, preferably 12 months or more, from first administration.

The methods and products of the invention concern dosages that are clinically proven safe and/or effective. Adverse events that impact whether the inventive dose, dosing regimen, treatment or method with rivaroxaban of the present invention are safe are adverse bleeding events that include, for example, major bleeding. “Major bleeding” as used herein is defined in accordance with ISTH criteria as clinically overt bleeding associated with a decrease in hemoglobin of g/dL, a transfusion of the equivalent of units of packed red blood cells or whole blood, or bleeding at a critical site, or with fatal outcome. Other adverse events include clinically relevant non-major bleeding and trivial (minimal) bleeding. “Clinically relevant non-major bleeding” as used herein is defined as clinically overt bleeding, which does not meet the criteria for major bleeding, but is associated with medical intervention, unscheduled contact with a physician, temporary cessation of treatment, discomfort for the patient, or impairment of activities of daily life.

The term “safe” as it relates to a dose, dosing regimen, treatment or method with rivaroxaban of the present invention refers to a dose, dosing regimen, treatment or method with rivaroxaban determined by the US Food and Drug Administration (“US FDA”) as acceptable for administration for thromboprophylaxis in pediatric patients, such as those 2 years and older, with congenital heart disease after the Fontan procedure, such as a dose, dosing regimen, treatment or method supported by evidence from a clinical trial measuring safety that has met the approval standards of the US FDA. For example, the clinical study may be a prospective, open-label, active controlled, multicenter study in pediatric patients to evaluate the single- and multiple-dose pharmacokinetic properties of rivaroxaban and the safety and efficacy of XARELTO® when used for thromboprophylaxis for up to 12 months in children with single ventricle physiology who had the Fontan procedure as described herein (Part B with 64 patients in the rivaroxaban group and 34 in the ASA, i.e., aspirin, group), that had one major bleeding event (1.6%) in the rivaroxaban group as compared to zero in patients in the aspirin group, and 4 (6.3%) clinically relevant non-major bleeding (CRNMB) events in the rivaroxaban group as compared to 3 (8.8%) in the aspirin group. In one such embodiment, the clinically proven safe dose, dosing regimen, treatment or method with rivaroxaban of the present invention is further shown as being safe by evidence from adequate and well-controlled studies of rivaroxaban in adults with additional pharmacokinetic, safety and efficacy data in studies in pediatric patients from birth to <18 years of age.

According to the invention, the terms “effective” or “efficacy,” as they relate to dose, dosing regimen, treatment or method with rivaroxaban of the present invention refer to efficacy for thromboprophylaxis in pediatric patients with congenital heart disease after a Fontan procedure. In certain embodiment, the pediatric patients are 2 years or older. Efficacy can be measured by the reduced risks or incidence of a thrombotic event (venous or arterial) after a Fontan procedure after the administration of rivaroxaban. As used herein the term “thrombotic event” is defined as the appearance of a new thrombotic burden within the cardiovascular system on either routine surveillance or clinically indicated imaging, or the occurrence of a clinical event known to be strongly associated with thrombus. Examples of a thrombotic event includes, such as, cardioembolic stroke, pulmonary embolism, thromboembolism. Preferably, the terms “effective” and efficacy” refer to a dose, dosing regimen, treatment or method with rivaroxaban of the present invention determined by the US FDA as acceptable for administration for thromboprophylaxis in pediatric patients, such as pediatric patients 2 years and older, with congenital heart disease after the Fontan procedure. In one embodiment, the clinically proven effective dose, dosing regimen, treatment or method with rivaroxaban of the present invention refers to a dose, dosing regimen, treatment or method with rivaroxaban of the present invention shown in the a prospective, open-label, active controlled, multicenter study in pediatric patients to evaluate the single- and multiple-dose pharmacokinetic properties of rivaroxaban and the safety and efficacy of XARELTO when used for thromboprophylaxis for up to 12 months in children with single ventricle physiology who had the Fontan procedure as described herein (Part B with 64 patients in the rivaroxaban group and 34 in the ASA group), showing that patients in the rivaroxaban group experienced fewer thromboembolic events than those treated with aspirin (for example, rivaroxaban 1 [1.6%] vs aspirin 3 [8.8%]). In one such embodiment, the clinically proven effective dose, dosing regimen, treatment or method with rivaroxaban of the present invention is further shown as being effective by evidence from adequate and well-controlled studies of rivaroxaban in adults with additional pharmacokinetic, safety and efficacy data in studies in pediatric patients from birth to <18 years of age.

In one embodiment, an amount clinically proven safe and clinically proven effective for thromboprophylaxis is the amount that when administered to a pediatric patient, such as a pediatric patient 2 years and older, with congenital heart disease after the Fontan procedure, results in a steady-state exposure of rivaroxaban at a geometric mean exposure AUC_{ss,24h} of 70% to 143% of AUC_{ss,24h} 1494 μg*h/L. In one embodiment, an amount clinically proven safe and clinically proven effective for thromboprophylaxis is the amount that when administered to a pediatric patient, such as a pediatric patient 2 years and older, with congenital heart disease after the Fontan procedure, results in a steady-state exposure of rivaroxaban at a geometric mean exposure AUC_{ss,24h} that is within the 2.5^{th} to 97.5^{th} percentile exposure range of the adult reference of AUC_{ss,24h} 1494 μg*h/L. In another embodiment, an amount clinically proven safe and clinically proven effective for thromboprophylaxis is the amount that when administered to a pediatric patient, such as a pediatric patient 2 years and older, with congenital heart disease after the Fontan procedure, results in a steady-state exposure of rivaroxaban at a geometric mean exposure AUC_{ss,24h} that is within the range of 1317 to 1576 μg*h/L.

Suitable dosages of rivaroxaban for use in the present invention include doses from 1.1 to 2.5 mg, including in particular, doses of 1.1, 1.6, 1.7, 2.0 or 2.5 mg, administered twice daily, depending on the body weight of the pediatric patient. Other dosages suitable for embodiments of the present invention include doses of 7.5 mg or 10 mg administered once daily.

As discussed herein and as will be appreciated, various forms, formulations and modes of administration are suitable for use in the methods herein. Preferably, rivaroxaban is administered orally, for example, as an oral suspension or a tablet. In certain embodiments, rivaroxaban in the form of granules is combined with water and mixed to form a resulting oral suspension, e.g. a 1 mg/mL suspension for use in the methods herein. In certain embodiments, rivaroxaban is administered as a tablet or as an oral suspension comprising rivaroxaban based on a body weight-adjusted dose, for example, as described herein below. In other embodiments, rivaroxaban is administered via naso-gastric or gastric administration of a suspension.

Durations of time will be based on a case-by-case basis and should include treatments beginning from one or more days post-Fontan procedure and continued as necessary.

In certain embodiments, rivaroxaban at an amount clinically proven safe and clinically proven effective for thromboprophylaxis is initially administered to the pediatric patient within 4 months after the Fontan procedure is completed. For example, rivaroxaban can be initially administered to the patient 1, 2, 3, 4, 5, 6, 7, 8, 9, 10, 11, 12, 13, 14, 15 or 16 weeks after the Fontan procedure is completed. Rivaroxaban can also be initially administered to the patient after 4 months of the completion of the Fontan procedure.

In certain embodiments, rivaroxaban at an amount clinically proven safe and clinically proven effective for thromboprophylaxis is administered to the pediatric patient once or twice daily, preferably for multiple days. In certain embodiment, rivaroxaban at an amount clinically proven safe and clinically proven effective for thromboprophylaxis is administered to the pediatric patient for a period of 1 week, 2 weeks, 3 weeks, 4 weeks, 5 weeks, 6 weeks, 7 weeks, 8 weeks or longer. In certain embodiments, rivaroxaban at an amount clinically proven safe and clinically proven effective for thromboprophylaxis is orally administered twice daily or once daily to a pediatric patient, such as a pediatric patient 2 years and older, after a Fontan procedure, preferably the initial administering is conducted within 4 months, such as within 1, 2, 3, 4, 5, 6, 7, 8, 9, 10, 11, 12, 13, 14, 15 or 16 weeks, after the Fontan procedure is completed, wherein the administering results in a steady-state exposure of rivaroxaban at a geometric mean exposure AUC_{ss,24h} of 70% to 143% of AUC_{ss,24h} 1494 μg*h/L.

In certain embodiments, rivaroxaban at an amount clinically proven safe and clinically proven effective for thromboprophylaxis is orally administered twice daily or once daily to a pediatric patient, such as a pediatric patient 2 years and older, after a Fontan procedure, wherein the administering results in a steady-state exposure of rivaroxaban at a geometric mean exposure AUC_{ss,24h} that is within the 2.5^{th} to 97.5^{th} percentile exposure range of the adult reference of AUC_{ss,24h} 1494 μg*h/L, for example, wherein the 2.5th to 97.5th percentile exposure ranges of the adult reference are 820 to 3216 μg*h/L for AUC24h,ss; 70.2 to 215.8 μg/L for Cmax,ss; and 3.55 to 52.0 μg/L for Ctrough,ss.

In certain embodiments, rivaroxaban at an amount clinically proven safe and clinically proven effective for thromboprophylaxis is orally administered twice daily or once daily to a pediatric patient, such as a pediatric patient 2 years and older, after a Fontan procedure, wherein the administering results in a steady-state exposure of rivaroxaban at a geometric mean exposure AUC_{ss,24h} that is within the range of 1317 to 1576 μg*h/L.

In certain embodiments of the present methods, the patient is a responder to said method of thromboprophylaxis as measured by the absence of the incidence of any thrombotic event in the patient for a period of 6 months or more, preferably 12 months or more, from first administration.

### EXAMPLES

A two-part study (Part A and Part B) was performed of rivaroxaban for thromboprophylaxis in children with CHD post-Fontan procedure.

Part A and Part B of Study

Patients

The sample size of 10 patients for Part A of the study was considered adequate for the initial assessment of the rivaroxaban PK in the studied pediatric patients to determine the dosing regimen to be evaluated in Part B. Eligible patients were boys or girls 2 to 8 years of age with single ventricle physiology and who completed the initial Fontan procedure within 4 months prior to enrollment, were considered to be clinically stable by the investigator and able to tolerate oral or enteral administration of a suspension formulation and oral/enteral feedings, and met the requirement of initial post-Fontan transthoracic echocardiographic screening with no reported thrombosis.

Study Design

This study was a prospective, open-label, active-controlled, multicenter study designed to evaluate the PK and PK/PD profiles, safety, and efficacy of rivaroxaban for thromboprophylaxis in pediatric patients 2 to 8 years of age with single ventricle physiology who have completed the Fontan procedure within 4 months prior to enrollment. Inclusion/exclusion criteria are provided in Table 1. The trial was approved by the institutional review board of each participating institution, as well as the appropriate national ethics committee. Written informed consent for trial participation was obtained from the parent or guardian of each patient. An Independent Data Monitoring Committee (IDMC) ensured patients' safety throughout the trial. A Central Independent Adjudication Committee (CIAC) reviewed all efficacy and safety events.

TABLE 1
UNIVERSE Inclusion/Exclusion criteria

Inclusion Criteria:

| | |
|---|---|
| 1. | Boys or girls 2 to 8 years of age with single ventricle physiology and who have completed the |
| | initial Fontan procedure within 4 months prior to enrollment |
| 2. | Considered to be clinically stable by the investigator and able to tolerate oral or enteral |
| | administration of a suspension formulation and oral/enteral feedings |
| 3. | Satisfactory initial post-Fontan transthoracic echocardiographic screening as defined in the |
| | Post-Fontan Echocardiographic Examination Research Protocol |
| 4. | Parent/legally acceptable representative must sign an informed consent form (ICF) and child |
| | assent will also be provided, if applicable, according to local requirements |

Exclusion Criteria:

| | |
|---|---|
| 1. | Evidence of thrombosis, including those that are asymptomatic confirmed by post-Fontan |
| | procedure transthoracic echocardiogram, or other imaging techniques, during the screening |
| | period of the study |
| 2. | History of gastrointestinal disease or surgery associated with clinically relevant impaired |
| | absorption |
| 3. | History of or signs/symptoms suggestive of protein-losing enteropathy |
| 4. | Active bleeding or high risk for bleeding contraindicating antiplatelet or anticoagulant therapy, |
| | including a history of intracranial bleeding |
| 5. | Criterion modified per Amendment INT-2 |
| 5.1 | Indication for anticoagulant or antiplatelet therapy other than current study, however: |
| | A subject who has received vitamin K antagonist (VKA) after the Fontan procedure may be |
| | eligible provided that the subject has discontinued VKA before the screening visit. Baseline |
| | laboratory samples must be obtained at least 7 days after the last dose of VKA. |
| | A subject who is receiving ASA at the time of the screening visit may be eligible and may |
| | continue receiving ASA provided the last dose is taken at least 24 hours prior to the first |
| | dose of study drug. |
| | A subject who is receiving heparin or LMWH after the Fontan procedure may be eligible and |
| | may continue receiving either of these anticoagulants during the screening period provided |
| | the study drug (rivaroxaban or ASA) is started 0 to 2 hours prior to the next scheduled |
| | administration of either of these anticoagulants and omit their administration thereafter. |
| 6. | Chronic use of nonsteroidal anti-inflammatory drugs (NSAIDs) |
| 7. | Platelet count <50 × 10⁹/L at screening |
| 8. | Criterion modified per Amendment INT-2 |
| 8.1 | Estimated glomerular filtration rate (eGFR) <30 mL/min/1.73 m² |
| 9. | Known clinically significant liver disease (eg, cirrhosis, acute hepatitis, chronic active hepatitis, or |
| | alanine aminotransferase (ALT) >3x upper limit of normal (ULN) with concurrent total |
| | bilirubin >1.5x ULN with direct bilirubin >20% of the total at screening) |
| 10. | Criterion modified per Amendment INT-2 |
| 10.1 | Known contraindication to ASA, or has or is recovering from chicken pox or flu-like symptoms |
| | (subjects participating in Part B only) |
| 11. | Criterion modified per Amendment INT-2 |
| 11.1 | Known allergies, hypersensitivity, or intolerance to rivaroxaban, ASA or its excipients |
| 12. | Inability to cooperate with study procedures |
| 13. | Combined P-glycoprotein (P-gp) and strong cytochrome P450 3A4 (CYP3A4) inhibitors (such as |
| | but not limited to ketoconazole, telithromycin, or protease inhibitors) use within 4 days before |
| | enrollment, or planned use during the study. Itraconazole use within 7 days before enrollment or |
| | planned use during the study. |
| 14. | Combined P-gp and strong CYP3A4 inducers (such as but not limited to rifampin/rifampicin, |
| | rifabutin, rifapentine, phenytoin, phenobarbital, carbamazepine, or St. John's Wort) use within |
| | 2 weeks before enrollment, or planned use during the study. |
| 15. | Planned use of drugs that are moderate CYP3A4 inhibitors (such as erythromycin) during the |
| | Initial PK, PD, and Safety Assessment Period of Part A only |
| 16. | Participation in a clinical study with an investigational drug or medical device in the previous 30 |
| | days prior to enrollment |
| 17. | Any condition for which, in the opinion of the investigator, participation would not be in the best |
| | interest of the subject (eg, compromise the well-being) or that could prevent, limit, or confound |
| | the protocol-specified assessments |
| 18. | Family member of an employee of the investigator or study site with direct involvement in the |
| | proposed study or other studies under the direction of that investigator or study site |

Part A of the study was the 12-month, non-randomized, open-label part of the study, which included a 12-day initial PK, PD, and Safety Assessment Period. An internal Data Review Committee (DRC) assessed by Day 12 the single- and multiple-dose rivaroxaban PK, PD, and the initial safety and tolerability data available from each patient, prior to the patient continuing in the study to complete the planned 12 months of open-label rivaroxaban therapy of Part A. The PK results from Part A are presented herein.

Part B was the randomized, open-label, active-controlled part of the study that evaluated the safety and efficacy of twice daily administration of rivaroxaban compared to ASA (usual standard of care) for thromboprophylaxis for 12 months. Patients randomized to rivaroxaban also had PK and PD assessments.

The transition from Part A to Part B was pre-defined based on an interim analysis of the Part A PK and PD data. If the geometric mean rivaroxaban exposures observed in Part A were within the predefined criteria of 70% to 143% (+/−30% on log scale) of the geometric mean adult reference exposure at 10 mg once daily (QD) in study ODIXa-HIP-OD (geometric mean AUC_{ss,24h} 1494 μg*h/L), Willmann S, et al. Pharmacokinetics of rivaroxaban in children using physiologically based and population pharmacokinetic modelling: an EINSTEIN-Jr phase I study. Thromb J. 16: 32 (2018), then the study would continue to Part B with the same dose regimen. Otherwise, the dose regimen was to be revised based on Part A observations, before starting enrollment into Part B.

For both Parts A and B, eligible patients were boys or girls 2 to 8 years of age with single ventricle physiology and who completed the initial Fontan procedure within 4 months prior to enrollment, were considered to be clinically stable by the investigator and able to tolerate oral or enteral administration of a suspension formulation and oral/enteral feedings, and met the requirement of initial post-Fontan transthoracic echocardiographic screening with no reported thrombosis. Patients were not eligible if there was evidence of thrombosis during the screening period or active bleeding or high risk of bleeding contraindicating antiplatelet or anticoagulant therapy. The use of vitamin K antagonists had to be discontinued before the screening visit and baseline laboratory assessments were to be obtained at least 7 days after the last dose of a vitamin K antagonist. Administration of ASA was permitted up to 24 hours before the first dose of the study drug. The use of heparin or any low molecular weight heparin was permitted if study drug was started 0 to 2 hours before the next scheduled dose of either of these anticoagulants and their administration was stopped thereafter. The use of combined P-gp and strong inhibitors or combined P-gp and strong inducers was not permitted within 4 days or 2 weeks, respectively, of enrollment.

Study Treatment

Rivaroxaban was administered twice daily (BID) in an open-label fashion as a 0.1% (1 mg/mL) oral suspension based on weight as shown in Table 2 (not related to prandial state). The pediatric dose regimen in the trial was designed to match the exposure range in adults treated with rivaroxaban 10 mg once daily, which is an effective dose for the prevention of thrombotic events in adults. The BID dosing regimen was selected to produce less fluctuation and a narrower concentration range (i.e., lower Cₘₐₓ and higher C_{trough}), relative to QD dosing, and increase the likelihood that pediatric exposures fell within the range observed in adults. Kubitza D, et al. Safety, pharmacodynamics, and pharmacokinetics of single doses of BAY 59-7939, an oral, direct factor Xa inhibitor. Clin Pharmacol Ther. 78: 412-21 (2005); Mueck W, et al. Clinical pharmacokinetic and pharmacodynamic profile of rivaroxaban. Clin Pharmacokinet. 53: 1-16 (2014).

TABLE 2
Dose Table for Rivaroxaban Administration in the Study

| Body weight | BID Dose ^{a} | Total Daily Dose ^{b} |
|---|---|---|
| [kg] | [mg or mL] | [mg] |
| 7 to <8 | 1.1 | 2.2 |
| 8 to <10 | 1.6 | 3.2 |
| 10 to <12 | 1.7 | 3.4 |
| 12 to <20 | 2 | 4 |
| 20 to <30 | 2.5 | 5 |

BID = twice daily;
kg = kilogram;
mg = milligram;
mL = milliliter
^{a} Oral suspension 0.1% (1 mg/mL).
^{b} Daily dose would provide exposure equivalent to exposure of 10 mg total daily dose in adults

Dose reduction due to impaired renal function was not required because children with an estimated glomerular filtration rate below 30 mL/min/1.73 m² were excluded from the study. Rivaroxaban was to be administered at approximately the same times each day in the morning and evening with 12-hour intervals.

Sample Analysis

Blood samples were collected for PK (i.e., plasma rivaroxaban concentrations) and full PD (prothrombin time [PT], activated partial thromboplastin time [aPTT], and anti-factor Xa activity) assessments on the first day of dosing between 0.5 to 1.5 hours and again between 1.5 to 4.0 hours postdose. Additional samples were collected on Day 4, just prior to rivaroxaban administration (PK, PT, aPTT) and again between 0.5 to 1.5 hours (PK, PT, aPTT), 1.5 to 4.0 hours (PK, PT, aPTT), and 6.0 to 8.0 hours (PK and full PD) postdose. At Month 3, samples were collected just prior to rivaroxaban dosing (PK and full PD) and at 0.5 to 1.5 hours postdose (PK, PT, aPTT) and 2.5 to 4 hours postdose (PK, PT, aPTT). An additional random sample (PK, PT, and aPTT) was collected at Month 12.

In Part B, blood samples were collected for assessment of PT and aPTT before the first dosing; PK and full PD samples were collected between 0.5 to 1.5 hours and again between 1.5 to 4.0 hours after the first dose. At Month 3, PK and full PD samples were collected just prior to rivaroxaban administration and again between 0.5 to 1.5 hours, and 2.5 to 4.0 hours postdose. An additional random sample (PK, PT, and aPTT) was collected at Month 12.

Blood samples for PK or PD assays were centrifuged, and the resulting plasma samples were frozen and stored below −15° C. until analyzed. Rivaroxaban plasma concentrations were determined using the validated liquid chromatography/mass spectrometry/mass spectrometry (LC-MS/MS) method with a lower limit of quantitation (LLOQ) of 0.5 μg/L. The pharmacodynamic effects of rivaroxaban were assessed by evaluation of prothrombin time (PT), activated partial thromboplastin time (aPTT), and anti-Factor Xa activity (AXA).

PT and aPTT were determined using an electromagnetic mechanical clot detection method at ARUP laboratory (Salt Lake City, Utah, USA). The PT assay was performed according to the manufacturers' instructions on the STA Compact Hemostasis Workstation (STA Compact coagulation analyzer) using one PT reagent (STA-Neoplastine CI Plus, Diagnostica Stago, Inc), with an International Sensitivity Index (ISI) of around 1.3. The primary read-out for PT was in seconds, as the International Normalization Ratio (INR) which allows for comparison of PT values regardless of the reagents and instruments used for its determination, is only calibrated and validated for VKA and cannot be used for any other anticoagulant without validation. The PT reportable range is between 10 and 120 seconds. STA PTTA agent was used for aPTT (Diagnostica Stago, Inc.) analysis. The read-out for aPTT is in seconds with a reportable range between 5 and 180 seconds. Anti-Factor Xa activity was determined at Eurofins Biomnis (France). Anti-Factor Xa activity was measured using a one-step reaction based on the inhibitory effect of rivaroxaban against factor Xa's activity of generating thrombin from prothrombin after adding rivaroxaban into the plasma-substrate mixture using the STA-COMPACT MAX2® system (Diagnostica Stago, Asnieres-sur-Seine, France). After the competitive reaction reached equilibrium, the quantity of paranitroaniline that is released in the system is inversely proportional to the concentration of rivaroxaban present in the test medium. The calibration range of the assay is 35 to 500 ng/mL, and the lower limit of quantification (LLOQ) is 35 ng rivaroxaban/mL.

Pharmacokinetics Sampling and Analysis

Patients in Part A received their first dose of rivaroxaban oral suspension on Day 1 (on site) and then continued taking rivaroxaban twice daily for 12 days (+9 days) to achieve steady state. PK and PD samples were collected on Day 1 and Day 4 (+2 days) of rivaroxaban administration (Table 3).

TABLE 3
Summary of Available Data to be Included in the Population PK Analysis of Part A

| | Day 1 (N) | Day 4 (N) | Month 3 (N) | Month 12 (N) |
|---|---|---|---|---|
| PK | postdose 0.5-1.5 h, (10) | Predose^{b}, (10) | Predose^{b}, (10) | 3 h predose to 8 h |
| Sampling^{a} | postdose 1.5-4 h (10) | postdose 0.5-1.5 h, (10) | postdose 0.5-1.5 h. (10) | postdose (10) |
| | | postdose 1.5-4 h, (10) | postdose 2.5-4 h (10) | |
| | | postdose 6.0-8 h, (10) | | |
| PD | postdose ^{c,} 0.5-1.5 h, | Predose^{b}, (10^{d}, 10^{e},) | predose^{b, c}, (7^{e}, 7^{e}, 3^{f}) | 3 h predose to 8 h |
| Sampling^{a} | (10^{d}, 10^{e}, 5^{f}) | postdose 0.5-1.5 h, (10^{d}, | postdose 0.5-1.5 h, | postdose (7^{d}, 7^{e}) |
| | postdose ^{c} 1.5-4 h | 10^{e}) | (8^{d}, 8^{e},) | |
| | (10^{d}, 10^{e}, 9^{f}) | postdose 1.5-4 h; (10^{d}, | postdose 2.5-4 h (7^{d,} 7^{e},) | |
| | | 10^{e}) | | |
| | | postdose ^{c} 6.0-8h, | | |
| | | (10^{d}, 10^{e}, 6^{f}) | | |

^{a}Sampling times are relative to morning dose,
^{b}Up to 3 hours predose,
^{c} Anti-Factor Xa activity collection;
^{d}sample numbers of PT;
^{e}sample numbers of aPTT;
^{f}sample numbers of Anti-Factor Xa activity
N: sample numbers
Study Part A contains total 100 PK samples, 89 PT samples, 89 aPTT samples, and 23 Anti-Factor Xa activity samples

After an internal data review committee assessment, the patients who were allowed to continue the 12-month treatment were also to have PK and PD samples collected at Month 3 and Month 12. Dose adjustments were made at Month 6 according to changes in the patient's body weight.

Exposure metrics of rivaroxaban, area under the plasma concentration-time curve from time 0 to 24 hours (AUC_{ss,24h}), maximum plasma concentration (C_{max,ss}), and concentration at the end of the dosing interval (C_{trough,ss}) at steady-state, were derived by a model from the Einstein Jr program as described in population PK section below. The current population PK analysis is intended to confirm if the exposure of rivaroxaban in the pediatric population in the UNIVERSE study can match that in the adult reference. For UNIVERSE Part A, individual empirical Bayesian post-hoc PK parameters were derived using the existing population PK model from the Einstein Jr program (with the NONMEM option: $ESTIMATION MAXEVAL=0).

Population PK Assessment

The population PK model used for the rivaroxaban PK profile evaluation was based on the Einstein Jr program. The pharmacokinetics of rivaroxaban were described by a linear two-compartment model with first-order oral absorption and first-order elimination from the central compartment with the absorption rate constant (KA), apparent clearance of the central compartment (CL/F), apparent central volume of distribution (Vc/F), apparent peripheral volume of distribution (Vp/F), and transport rate between the central and peripheral compartments (CI) as structural model parameters. CL/F, Q/F, Vp/F, and Vc/F were fig. allometrically scaled with body weight. Inter-individual variability (IIV) was identified for KA and CL/F. Residual error was described by a proportional error model. The known dose-dependency of relative bioavailability (F) was described in the population PK model by using the previously reported bioavailability function in adults after replacing the absolute dose in mg by dose/weight (DOSE/WGHT) ratio (see Equation 1) using the parameter estimates identified for adult population (Fₘᵢₙ=0.590, Fₘₐₓ=1.25, D50=14.4 mg). Willmann S, et al. Integrated Population Pharmacokinetic Analysis of Rivaroxaban Across Multiple Patient Populations. CPT Pharmacometrics Syst Pharmacol. 7: 309-20 (2018).

$$F = {F}_{min} + ( {F}_{max} - {F}_{min} ) \cdot {e}^{- \frac{{log}_{e} ( 2 )}{D 50} \cdot \frac{DOSE}{WGHT}} \qquad ( 1 )$$

For WGHT, the same median (82.48 kg) of body weight was used as in the integrated population PK model. Exposure metrics of rivaroxaban, the area under the plasma concentration-time curve from time 0 to 24 hours at steady-state (AUC_{ss,24h}), the maximum plasma concentration at steady-state (C_{max,ss}), and the concentration at the end of the dosing interval at steady-state (C_{trough,ss}), were derived using population post hoc parameters. Lensing AWA, et al. Rivaroxaban versus standard anticoagulation for acute venous thromboembolism in childhood. Design of the EINSTEIN-Jr phase III study. Thromb J. 16: 34 (2018); Monagle P, et al. Bodyweight-adjusted rivaroxaban for children with venous thromboembolism (EINSTEIN-Jr): results from three multicentre, single-arm, phase 2 studies. The Lancet Haematology. 6: e500-e9. (2019). Individual exposure results were plotted as a function of bodyweight and compared with the adult reference range (2.5^{th}-97.5^{th} percentile prediction range obtained through using the model to perform exposure simulations in 1000 virtual patients of HIP and Knee replacement) and the pediatric reference range (2.5^{th}-97.5^{th} percentile prediction range obtained using the model to perform exposure simulations in 1000 virtual patients in the EINSTEIN Jr program assuming the UNIVERSE study weight based dosing regimen and with bodyweight range from 7 to 30 kg) Turpie A G, et al. BAY 59-7939: an oral, direct factor Xa inhibitor for the prevention of venous thromboembolism in patients after total knee replacement. A phase II dose-ranging study. Journal of thrombosis and haemostasis: JTH. 3: 2479-86 (2005).

Model Qualification

Diagnostic plots of observed data vs. population prediction (PRED) and observed data vs. individual predictions (IPRED) were examined for adequate fit. Plots of conditional weighted residuals (CWRES) vs. PRED and CWRES vs. time (times after last and first doses) were inspected for evidence of systematic lack of fit, and to confirm the absence of bias in the error distributions. Individual deviations from the population mean were expected to be normally distributed with a mean of zero and variance σ².

PBPK Modeling and Simulation

A first PBPK model for rivaroxaban administration to healthy children was established previously. (see, Willmann S, et al. Development of a paediatric population-based model of the pharmacokinetics of rivaroxaban. Clin Pharmacokinet. 2014; 53: 89-102; Willmann S, et al. Pharmacokinetics of rivaroxaban in children using physiologically based and population pharmacokinetic modelling: an EINSTEIN-Jr phase I study. Thromb J. 2018; 16: 32.) This model was scaled from the PBPK model for adults by accounting for: 1) the age-dependency of anthropometric, anatomical, and physiological parameters (e.g., body weight, height, organ volumes, blood flow rates, and tissue composition), 2) the ontogeny and variability of active processes that are relevant for rivaroxaban pharmacokinetics (PK) (e.g., CYP3A4 activity, transporter activity), and drug-related parameters (e.g., protein binding). This model served as a basis for the development of a pediatric Fontan-PBPK model which considered relevant pathophysiological conditions of post-Fontan patients such as a reduced cardiac output and a lower body weight for a given age compared to healthy children. With this Fontan-PBPK model, seven virtual pediatric post-Fontan populations for each age (2, 3, 4, 5, 6, 7, 8 years) comprising 2,000 individuals per gender were generated to predict the relationship between dose and exposure of rivaroxaban in post-Fontan patients in comparison to healthy children.

PK/PD Assessment

Individual results of PT and aPTT were plotted as a function of plasma concentration of rivaroxaban and compared with the pediatric reference range (90% prediction range obtained using the model to perform exposure simulations in 1000 virtual patients in the EINSTEIN Jr program assuming the UNIVERSE study weight based dosing regimen and with bodyweight range from 7 to 30 kg) and the adult reference range (PT 90% prediction range obtained using the model to perform exposure simulations in 1000 virtual patients of HIP and Knee replacement studies who had received 10 mg rivaroxaban total daily dose. Individual results of Anti-Factor Xa activity were plotted as a function of concentration of rivaroxaban

Computer Software

For the analyses, non-linear mixed effect modeling of concentration-time data was performed using NONMEM (Version 7.3, Icon Development Solutions, Ellicott City, Md., USA) (25). The first-order condition estimation approximation (FOCE) was used as an estimation method. Furthermore, the INTERACTION option was used in NONMEM, which takes into account the presence of an interaction between the two levels of random effects. Small modifications to the analysis dataset, exploratory analysis, diagnostic graphics and post-processing of NONMEM analysis results were carried out using R (version 3.4.2, The R foundation for Statistical Computing) Diagnostic graphics, exploratory analyses, and post-processing of NONMEM output, R and RStudio (Version 1.1.383, RStudio Inc, Boston, USA) and/or the Pirana software package (version 2.9.7, Pirana Software and Consulting BV, Denekamp, the Netherlands).

Exposure-Efficacy and Exposure-Safety Assessment

The association between rivaroxaban exposure and thrombosis events was explored visually using box plots. Similarly, the association between rivaroxaban exposure and bleeding events was explored visually using box plots.

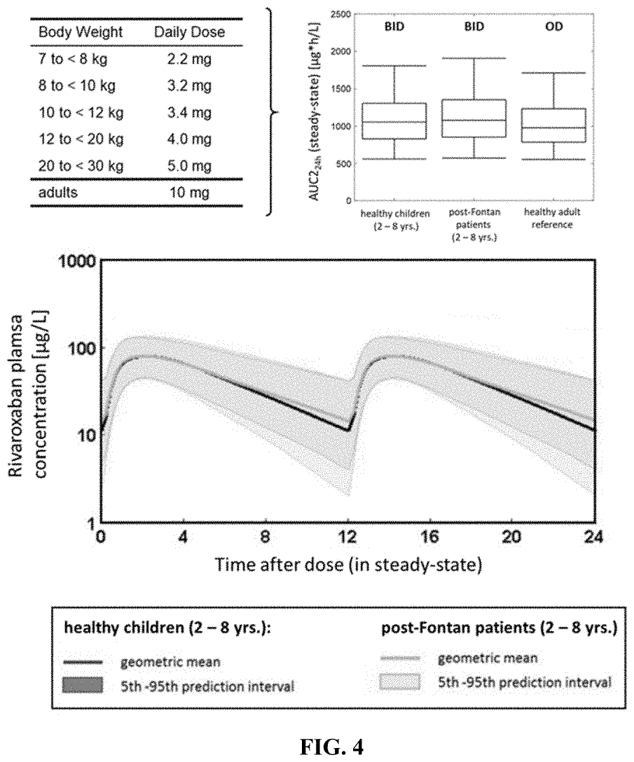

The relationship between dose and exposure that was predicted by the Fontan-PBPK model for virtual post-Fontan patients 2 to 8 years of age was practically identical to the relationship that was predicted for weight-matched healthy children (based on the initial PBPK model), indicating that the known pathophysiological conditions that were reflected in the Fontan-PBPK model do not alter the PK of rivaroxaban, in particular the important CL/F metric. The PBPK model simulation suggested a body weight-based dose regimen in pediatric patients post-Fontan procedure which was expected to produce rivaroxaban exposures similar to the adult reference range at 10 mg dose. FIG. 4 shows the predicted steady-state plasma concentration-time course of rivaroxaban for post-Fontan patients receiving the proposed body weight-adjusted dosing scheme in comparison to healthy children receiving the same dose. This dose regimen was then incorporated and tested in the UNIVERSE study.

Part A of the Study

A total of 12 patients (ages 2-8 years) were enrolled in UNIVERSE Part A, with 10 patients completing the PK/PD assessments for 12 months. Since all 12 patients had post-dose PK samples taken, they are all included in Part A PK assessment.

Pharmacokinetics

The majority of the observed plasma concentrations of rivaroxaban in UNIVERSE Part A were within the 90% CI range from both models, indicating the plasma concentrations time profiles were adequately described by the model from the Einstein Jr program and from the adult HIP and KNEE replacement studies.

Additionally, the model predicted the AUC_{ss,24h} values; the C_{max,ss}, and the C_{trough,ss} at steady-state were compared with the 2.5^{th} to 97.5^{th} percentile range of model prediction from both the pediatric model and the adult model. Relative oral bioavailability decreased with increasing dose per body weight. Individual values for AUC_{ss,24h}, C_{max,ss}, and C_{trough,ss} were within the adult reference range, irrespective of age, bodyweight, and treatment regimens. Most of the individual values were within the 2.5^{th} to 97.5^{th} percentile range of the adult exposure range.

These results of concentration-time profiles as well as exposure of rivaroxaban in post-Fontan procedure pediatric patients from Part A strongly indicate that the bodyweight-adjusted BID dosing regimen achieves exposures in children similar to the exposures in adults who received 10 mg total daily dose.

Pharmacodynamics

PT and aPTT results from UNIVERSE Part A are well predicted by the Einstein Jr program PKPD model, indicating that PKPD profiles of rivaroxaban in children after the Fontan procedure are similar to those in the Einstein Jr program population. Furthermore, the PT results from UNIVERSE Part A are also consistent with the previous adult model. All individual PT data points were within the prediction limits of both adult data and Einstein Jr program data, indicating no apparent difference between pediatric and adult studies in the correlation between PT and rivaroxaban plasma concentrations.

The correlation graphs for aPTT were less steep than for PT, due to a lower sensitivity of the aPTT assay to rivaroxaban compared with the PT assay. Anti-factor Xa correlated strongly with rivaroxaban plasma concentrations (r=0.932) in UNIVERSE Part A.

Discussion

Part A of the study showed that a bodyweight-based dose regimen of rivaroxaban in pediatric patients after the Fontan procedure provides comparable plasma exposure to that observed in adults receiving 10 mg total daily dose, the dose used for prevention of VTE in adults after they had major orthopedic surgery or after hospitalization of medically ill adult patients. In Part A, most of the observed PK concentrations were within the 90% prediction interval of the Einstein Jr program model as well as the adult reference ranges.

The pharmacodynamic and pharmacokinetic data from Part A showed good correlation between these parameters, particularly between rivaroxaban concentrations and Anti-Factor Xa activity tests. Anti-Factor Xa activity followed a linear relationship with the change of rivaroxaban concentration which is also observed in adult patients with non-valvular atrial fibrillation.

A total of 12 patients (ages 2-8 years) were enrolled in UNIVERSE Part A, with 10 patients completing the PK and PD assessments for 12 months. Since all 12 patients had post-dose PK samples taken, they are all included in Part A PK assessment. An interim examination of the Part A PK run-in results of the 12 patients showed that the rivaroxaban exposures were in a similar range as those observed in adult reference. The steady-state exposure of rivaroxaban with the body weight-based dose regimen achieved a geometric mean exposure AUC_{ss,24h} of 1698 (90% confidence interval [CI]: 1336, 2157) μg*h/L in Part A, which was within the predefined range of 70% to 143% of the adult reference exposure at 10 mg QD dose in the study ODIXa-HIP-OD (geometric mean AUC_{ss,24h} 1494 μg*h/L). A noted difference in the steady state C_{trough} was due to different dosing intervals used in adults (QD) compared to pediatric patients (BID). In addition, the PT and aPTT results observed in Part A were also within the 99% prediction range observed in the adult reference studies ODIXa-HIP2 and ODIXa-KNEE. Results are shown in Table 4. Based on these data, the body weight-based dose regimen proposed by the post-Fontan PBPK model was considered appropriate and the UNIVERSE study progressed into Part B with the same dose regimen.

In conclusion, in Part A, the bodyweight-adjusted dosing regimen of 10 mg equivalent rivaroxaban dosing in pediatric patients after the Fontan procedure was demonstrated to provide comparable exposure to 10 mg total daily dose in adults with similar AUC_{ss,24h}, C_{max,ss} and C_{trough,ss}. The favorable pharmacodynamic and pharmacokinetic profiles of rivaroxaban provided the foundation for the dosing schedule in Part B, the randomized and active-controlled part of the study, for the prevention of thromboembolic events in the pediatric population. Results of Part B of the study are provided below.

TABLE 4
Comparison of rivaroxaban exposures between the UNIVERSE
study and the Adult Reference Study ODIXa-HIP OD

| | | UNIVERSE Part A | UNIVERSE all patients | Adult |
|---|---|---|---|---|
| | | (weight-based dose | (weight-based dose | Reference |
| Variables | Exposure Metrics | regimen, BID) | regimen, BID) | (10 mg QD) |
| N | | 12 | 76 | 140 |
| AUC_{24 h, ss} | Geometric | 1698 (1336, | 1440 (1317, | 1494 (1425, |
| | Mean (90% CI) | 2157) | 1576) | 1565) |
| | Median (Range) | 1718 (776.8, | 1477 (484.2, | 1452 (565.4, |
| | | 4444) | 4444) | 4747) |
| | Geometric mean ratio | | 0.96 (0.87, 1.07) | |
| | UNIVERSE/Adult (90% CI) | | | |
| C_{max, ss} | Geometric | 123.9 (102.4, | 109.0 (100.4, | 125.8 (120.6, |
| | Mean (90% CI) | 149.9) | 118.5) | 131.31 |
| | Median (Range) | 121.8 (54.8, | 113.3 (39.7, | 127.6 (54.0, |
| | | 265.2) | 287.1) | 292.8) |
| | Geometric mean ratio | | 0.87 (0.79, 0.95) | |
| | UNIVERSE/Adult (90% CI) | | | |
| C_{trough, ss} | Geometric | 28.6 (20.2, | 22.8 (20.4, | 13.9 (12.6, |
| | Mean (90% CI) | 40.6) | 25.5) | 15.4) |
| | Median (Range) | 29 (8.6, | 23.2 (6.4, | 14.3 (0.8, |
| | | 104.7) | 304.7) | 99.3) |

| | |
|---|---|
| Geometric mean ratio | 1.64 (1.41, 1.90) |
| UNIVERSE/Adult (90% CI) | |

AUC_{24 h, ss} = area under the plasma concentration-time curve to 24 hours at steady-state; BID= twice daily; CI = confidence interval; C_{max, ss} = post-dose maximum concentration at steady-state; C_{trough, ss} = predose concentrations at steady-state; QD = once a day.

Part B of Study

Randomization

In Part A, the subjects were not randomized. All subjects received rivaroxaban. In Part B, Central randomization began once the cumulative subjects' data from initial PK and Safety Assessment in Part A were deemed acceptable by the IDMC. The assignment to treatment groups was 2:1 (rivaroxaban:aspirin).

TABLE 5
Dose Table for Rivaroxaban Administration in Part B

1 mg XARELTO = 1 mL suspension

| | Dosage | |
|---|---|---|
| Bodyweight | 2 times a day^{†} | Total daily dose* |
| 7 to <8 kg | 1.1 mg | 2.2 mg |
| 8 to <10 kg | 1.6 mg | 3.2 mg |
| 10 to <12 kg | 1.7 mg | 3.4 mg |
| 12 to <20 kg | 2.0 mg | 4.0 mg |
| 20 to <30 kg | 2.5 mg | 5.0 mg |

*All doses can be taken with or without food
^{†}2 times a day: approximately 12 hours apart

Results from Part A and Part B

Baseline Characteristics

Among the 112 subjects enrolled, 66 (58.9%) subjects were male, and 68 (60.7%) subjects were white. The mean age was 3.9±1.74 years, and the median age was 4 years. The demographic and baseline characteristics were generally balanced between the rivaroxaban and the aspirin groups in Part B; however, there were slightly more males (67.6%) and slightly shorter duration between the Fontan procedure and first study drug dose (median 24 days) in the aspirin group than in the rivaroxaban group (54.5% males and a median of 34 days between the Fontan procedure and first dose). The rivaroxaban Part A group had a younger mean age (2.5+/−0.67), a lower mean weight and a shorter mean duration between the Fontan procedure and first dose than both groups in Part B. The marked shorter duration in days between the Fontan procedure and the first dose of study drug in Part A was probably due to the study design which required frequent blood draws on Day 1 and Day 4 for PK/PD testing in Part A. Most investigators wanted to have Day 1 and Day 4 performed while the children were still hospitalized after the Fontan procedure and with an intravenous catheter still in place, to avoid sticking the child multiple times for the blood draws.

Disposition

A total of 129 children with single ventricle physiology were screened at 36 sites in 10 countries (Argentina, Belgium, Brazil, Canada, Japan, Malaysia, Mexico, the Netherlands, Spain and the United States). Of them, 112 were enrolled in the UNIVERSE study (12 in the rivaroxaban Part A group, 66 in the rivaroxaban Part B group and 34 in the aspirin Part B group).

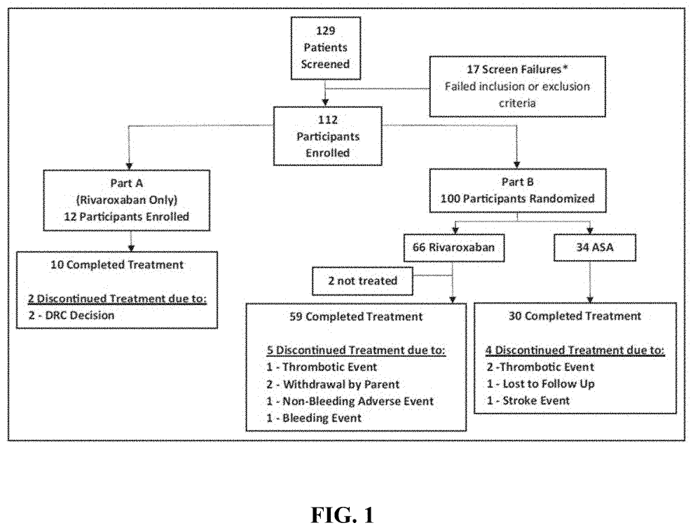

Of the 112 subjects enrolled, 107 (95.5%) subjects completed the study (11 [91.7%] subjects in the rivaroxaban Part A group, 63 [95.5%] in the rivaroxaban Part B group, and 33 [97.1%] in the aspirin Part B group). A total of 110 children received at least one dose of study drug and were included in the full/safety analysis set. See FIG. 1.

Of the 110 participants who received study drug, 99 (90.0%) subjects completed study treatment (10 [83.3%] subjects in the rivaroxaban Part A group, 59 [92.2%] in the rivaroxaban Part B group, and 30 [88.2%] in the aspirin Part B group), and 11 (10.0%) subjects discontinued study treatment prematurely (2 [16.7%] subjects in the rivaroxaban Part A group, 5 [7.8%] in the rivaroxaban Part B group, and 4 [11.8%] in the aspirin Part B group). In Part B, the most frequent reason for premature discontinuation of study treatment was thrombosis for 3 (2.7%) subjects (1 [1.6%] in the rivaroxaban group, and 2 [5.9%] in the aspirin group) followed by withdrawal by parent or guardian, 2 (1.8%) subjects in the rivaroxaban group. In Part A, the most frequent reason for premature discontinuation of study treatment was a DRC decision (2 [16.7%] in the rivaroxaban Part A group). Two subjects were withdrawn from Part A of the study by DRC decision, due to a steady state AUC above the pre-specified upper threshold of the target AUC range. These subjects did not have any bleeding or a reported adverse event while they were in the study. Only 1 subject had the study treatment discontinued due to bleeding (the subject in the rivaroxaban Part B group with major bleeding event).

Extent of Exposure

For Part B, 12 months of treatment was considered 360 days ±7 days.

The median total duration of treatment exposure in the rivaroxaban Part A group, the rivaroxaban Part B group and the aspirin Part B group was 359 days.

Overall, 94 (85.5%) subjects had 353 days of treatment exposure and 99 (90.0%) subjects had 300 days of treatment exposure (exceeding an FDA written request requirement).

Efficacy

Primary efficacy outcome: Any thrombotic event, venous or arterial, symptomatic or asymptomatic defined as:
The appearance of a new thrombus within the cardiovascular system on either routine surveillance or clinically indicated imaging, or
The occurrence of a clinical event known to be strongly associated with thrombus (e.g., stroke, pulmonary embolism).

The number and proportion of subjects with the primary efficacy outcome in Part B were favorable to the rivaroxaban group (1 [1.6%]) vs. the aspirin group (3 [8.8%]). In the rivaroxaban group the single event reported was 1 (1.6%) pulmonary embolus on day 84 of the study treatment (121 days post Fontan procedure), and, in the aspirin group, the 3 events were 2 (5.9%) venous thrombotic events on Days 177 and 179 of treatment (191 and 183 days post Fontan procedure, respectively) and 1(2.9%) ischemic stroke on day 122 of treatment (133 days post Fontan procedure).

There was 1 venous thrombotic event in the rivaroxaban Part A group on day 362 of treatment (364 days post Fontan procedure).

All efficacy outcomes were adjudicated by the CIAC, blinded to assigned treatment.

There were no primary efficacy outcomes reported after Month 12 or after the early discontinuation of study drug.

There was a trend for less thromboembolic events in the Rivaroxaban group.

The results of the primary efficacy are shown in FIG. 2 (Full Set, Part B Only) and Table 6.

TABLE 6
Summary of Primary Efficacy Outcome
Summary of Primary Efficacy Outcome, Up-to-End-of
Treatment by CIAC; Full Analysis Set

| | | Rivaroxaban | | Aspirin | |
|---|---|---|---|---|---|
| | Part A | Part B | Total | Part B | Total |
| Analysis Set: Full | 12 | 64 | 76 | 34 | 110 |
| Primary Efficacy Outcome | 1 (8.3%) | 1 (1.6%) | 2 (2.6%) | 3 (8.8%) | 5 (4.5%) |
| Ischemic Stroke | 0 | 0 | 0 | 1 (2.9%) | 1 (0.9%) |
| Pulmonary Embolism | 0 | 1 (.6%) | 1 (1.3%) | 0 | 1 (0.9%) |
| Venous thrombosis | 1 (8.3%) | 0 | 1 (1.3%) | 2 (5.9%) | 3 (2.7%) |
| Arterial/intracardiac | 0 | 0 | 0 | 0 | 0 |
| Thrombosis | | | | | |
| Other Thrombosis | 0 | 0 | 0 | 0 | 0 |

Note:
Full Analysis Set: all subjects in Part A who receive at least 1 dose of study agent and all subjects in Part B who are randomized and receive at least 1 dose of study agent.
Note:
Up-to-End-of Treatment is defined as the period starting from the first dose of study agent to end of treatment visit.
Note:
Percentages calculated with the number of subjects in each group as denominator.
Note:
CIAC = Central Independent Adjudication Committee.

In the rivaroxaban Part B group, 1 participant (2%) was reported with pulmonary embolism on Day 84 of study treatment (121 days post Fontan procedure). In the ASA group, 3 participants (9%) were reported with thrombotic events (2 participants (6%) with venous thrombotic events reported on Day 177 and Day 179 of treatment [191 days and 183 days post Fontan procedure, respectively] (the Day 179 event being a thrombosis in the conduit); and 1 participant (3%) who had an ischemic stroke on Day 122 of treatment [133 days post Fontan procedure]). In the rivaroxaban Part A group, 1 participant (8%) had a venous thrombotic event on Day 362 of treatment (364 days post Fontan procedure) (Table 6b). Overall, the rivaroxaban group had a proportion of thrombotic events of 3% vs 9% in the ASA group (Table 6b).

TABLE 6b

| | Rivaroxaban | | | ASA |
|---|---|---|---|---|
| | Part A | Part B | Total | Part B |
| | N = 12 | N = 64 | N = 76 | N = 34 |
| Primary efficacy outcome: | 1 (8%) | 1 (2%) | 2 (3%) | 3 (9%) |
| Any thrombotic event | | | | |
| Ischemic stroke | 0 | 0 | 0 | 1 (3%) |
| Pulmonary embolism | 0 | 1 (2%) | 1 (1%) | 0 |
| Venous thrombosis | 1 (8%) | 0 | 1 (1%) | 2 (6%) |
| Arterial/intracardiac thrombosis | 0 | 0 | 0 | 0 |

ASA = acetylsa licylic acid
Note:
Full Analysis Set: all participants in Part A who received at least 1 dose of study drug and all participants in Part B who were randomized and received at least 1 dose of study drug.
Note:
Percentages were calculated with the number of participants in each group as denominator

Safety

The primary safety outcome was ISTH defined major bleeding. Major bleeding was defined as overt bleeding and:

1) Associated with a fall in hemoglobin of 2 g/dL or more; or 2) Leading to a transfusion of the equivalent of 2 or more units of packed red blood cells or whole blood in adults; or 3) Occurring in a critical site: intracranial, intraspinal, intraocular, pericardial, intraarticular, intramuscular with compartment syndrome, retroperitoneal; or 4) Contributing to death.

The secondary safety outcomes were: Clinically relevant non-major bleeding and trivial (minimal) bleeding events.

Other safety outcomes: Any adverse events and serious adverse events (SAES).

Primary safety outcome (Major bleeding)

Major bleeding events as adjudicated by the CIAC were numerically less in the aspirin group than in the rivaroxaban Part B group (0 vs 1 (1.6%). The single event of major bleeding reported in the rivaroxaban group occurred in a non-critical site, epistaxis, which required transfusion. The subject with major bleeding was discontinued from study treatment per protocol, due to meeting the primary safety outcome.

No major bleeding events were reported in the rivaroxaban Part A group.

Secondary Safety Outcomes:

1. Clinically relevant non-major bleeding (CRNMB)

In Part B, there were 4 [6.3%] CRNMB events in the rivaroxaban as compared to 3 [8.8%] in the aspirin group.

CRNMB events reported in the rivaroxaban group occurred in the lower gastrointestinal tract (2 [3.1%]), gingival (1 [1.6%]), and the skin (1 [1.6%]). In the aspirin group CRNMB occurred in the lower gastrointestinal tract (1 [2.9%]), the skin (1 [2.9%]), hematoma (1 [2.9%]), and subconjunctival (1 [2.9%]).

In the rivaroxaban Part A group there was 1 (8.3%) subject reported with CRNMB (site of bleeding was in the skin).

### 2. Trivial Bleeding

In Part B, the rate of trivial bleeding was comparable in the rivaroxaban and the aspirin groups (21 [32.8%] vs. 12 [35.3%] respectively). The most frequent site of trivial bleeding was skin in both groups (14 [21.9%] in the rivaroxaban group and 8 [23.5%] in the aspirin group.

In the rivaroxaban Part A group there were 3 (25.0%) subjects reported with trivial bleeds (the most frequently reported was hematoma, 2 [16.7%]).

### 3. Any Bleeding

In Part B, the rate of subjects with any bleeding events was similar in the rivaroxaban group than the aspirin group (23 [35.9%]) in the rivaroxaban vs. 12 [41.2%] in the aspirin group).

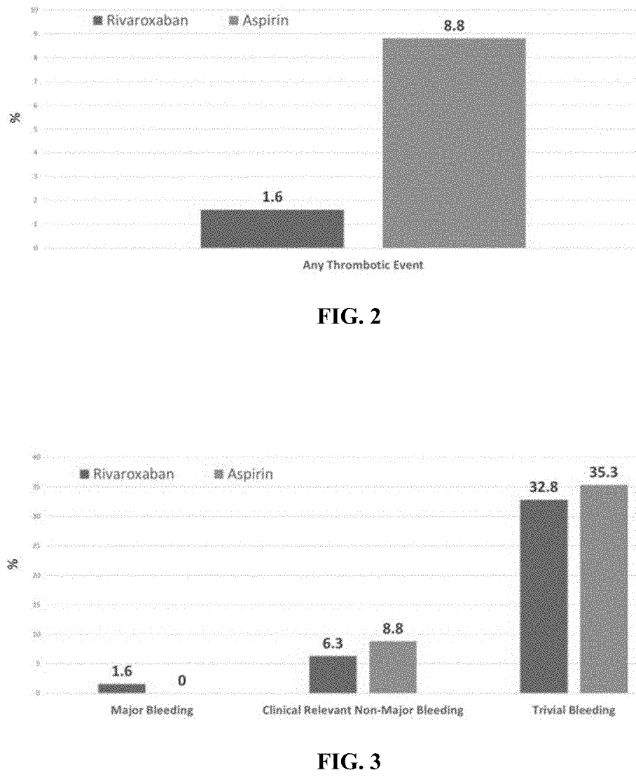

The results of the safety outcomes are shown in FIG. 3 (Safety Set, Part B Only) and Table 7.

TABLE 7
Summary of Bleeding Events
TSFBL01B: Summary of Bleeding, On-treatment as Adjudicated by CIAC; Safety Analysis Set

| | Rivaroxaban | | | | | | Aspirin | | | |
|---|---|---|---|---|---|---|---|---|---|---|
| | Part A | | Part B | | Total | | Part B | | Total | |
| Analysis set: Safety | 12 | | 64 | | 76 | | 34 | | 110 | |
| Subjects with 1 or | 4 | (33.3%) | 23 | (35.9%) | 27 | (35.5%) | 14 | (41.2%) | 41 | (37.3%) |
| more on- treatment | | | | | | | | | | |
| bleeding events | | | | | | | | | | |
| Major Bleeding | 0 | | 1 | (1.6%) | 1 | (1.3%) | 0 | | 1 | (0.9%) |
| Clinically relevant non- | 1 | (8.3%) | 4 | (6.3%) | 5 | (6.6%) | 3 | (8.8%) | 8 | (7.3%) |
| major bleeding | | | | | | | | | | |
| Gastrointestinal | 0 | | 2 | (3.1%) | 2 | (2.6%) | 1 | (2.9%) | 3 | (2.7%) |
| GI-Lower | 0 | | 2 | (3.1%) | 2 | (2.6%) | 1 | (2.9%) | 3 | (2.7%) |
| Gingival | 0 | | 1 | (1.6%) | 1 | (1.3%) | 0 | | 1 | (0.9%) |
| Hematoma | 0 | | 0 | | 0 | | 1 | (2.9%) | 1 | (0.9%) |
| Skin | 1 | (8.3%) | 1 | (1.6%) | 2 | (2.6%) | 1 | (2.9%) | 3 | (2.7%) |
| Subconjunctival | 0 | | 0 | | 0 | | 1 | (2.9%) | 1 | (0.9%) |
| Trivial bleeding | 3 | (25.0%) | 21 | (32.8%) | 24 | (31.6%) | 12 | (35.3%) | 36 | (32.7%) |
| Epistaxis | 0 | | 7 | (10.9%) | 7 | (9.2%) | 3 | (8.8%) | 10 | (9.1%) |
| Gastrointestinal | 0 | | 1 | (1.6%) | 1 | (1.3%) | 1 | (2.9%) | 2 | (1.8%) |
| GI-Lower | 0 | | 0 | | 0 | | 1 | (2.9%) | 1 | (0.9%) |
| GI-Upper | 0 | | 1 | (1.6%) | 1 | (1.3%) | 0 | | 1 | (0.9%) |
| Gingival | 1 | (8.3%) | 3 | (4.7%) | 4 | (5.3%) | 1 | (2.9%) | 5 | (4.5%) |
| Hematoma | 2 | (16.7%) | 7 | (10.9%) | 9 | (11.8%) | 2 | (5.9%) | 11 | (10.0%) |

| | | | | | | | | | |
|---|---|---|---|---|---|---|---|---|---|
| Skin | 0 | 14 | (21.9%) | 14 | (18.4%) | 8 | (23.5%) | 22 | (20.0%) |

| | | | | | | | | |
|---|---|---|---|---|---|---|---|---|
| Vascular Access Site | 0 | 2 | (3.1%) | 2 | (2.6%) | 0 | 2 | (1.8%) |

Note:
Percentages calculated with the number of subjects in each group as denominator.
Note:
Incidence is based on the number of subjects not the number of events. A subject may appear in different sites/categories.
Note:
On-treatment is defined as the period starting from the first dose of study agent to 2 days after the last dose of the study agent administration inclusively.
Note:
Safety Analysis Set: all subjects in Part A who receive at least 1 dose of study agent and all subjects in Part B who are randomized and receive at least 1 dose of study agent.
Note:
The primary safety outcome is major bleed that meets the ISTH definition. ISTH = International Society on Thrombosis and Haemostasis.
Note:
The only major bleeding occurred in non-critical site - Epistaxis.
Note:
Major bleeding is defined as overt bleeding and: 1) Associated with a fall in hemoglobin of 2 g/dL or more; or 2) Leading to a transfusion of the equivalent of 2 or more units of packed red blood cells or whole blood in adults; or 3) Occurring in a critical site: intracranial, intraspinal, intraocular, pericardial, intraarticular, intramuscular with compartment syndrome, retroperitoneal; or 4) Contributing to death.
Note:
CIAC = Central Independent Adjudication Committee.

Other adverse events (including bleeding events): There were 11 (91.7%), 55 (85.9%) and 29 (85.3%) subjects who experienced at least one treatment-emergent adverse event and 6 (50.0%), 18 (28.1%) and 8 (23.5%) subjects who experienced at least one treatment-emergent serious adverse event in the rivaroxaban Part A group, in the rivaroxaban Part B group, and in the aspirin Part B group, respectively.

In Part B, adverse events and serious adverse events were generally balanced between the rivaroxaban and the aspirin groups except for pleural effusions, which were more frequently reported in the rivaroxaban group than in the aspirin group (12 [18.8%] vs. 2 [5.9%]). Adverse events were more frequently reported in the infections and infestations system organ class (SOC) in both groups (40 [62.5%] in the rivaroxaban group and 22 [64.7%] in the aspirin group). There were 2 (3.1%) subjects permanently discontinued from treatment for a treatment emergent adverse event in the rivaroxaban group, 1 (1.6%) subject due to reaching the primary safety outcome (major bleed) and was discontinued as per protocol and 1 (1.6%) subject due to the adverse event of having mood disturbance and the parent withdrew her consent.

In Part A, infections and infestations was also the system organ class with more frequently reported AEs (8 [66.7%]).

AE/SAE profiles were as expected from this patient population and from rivaroxaban's AE profile in adults. There were no new safety signals uncovered for rivaroxaban.

Deaths—No subjects died during the study.

Tables 8-10 provide a summary of adverse events—safety analysis set

TABLE 8
Summary of Adverse Events

| | Rivaroxaban | | Aspirin | |
|---|---|---|---|---|
| | Part A | Part B | Part B | |
| | N = 12 | N = 64 | N = 34 | |
| All TEAEs | 11 (91.7%) | 55 (85.9%) | 2.9 | (85.3%) |
| All TESAEs | 6 (50.0%) | 18 (28.1%) | 8 | (23.5%) |
| TEAEs resulting in | 0 | 2 (3.1%) | 0 | |
| Permanent Discontinuation | | | | |
| of Study Agent | | | | |
| AEs related to Study Agent | 3 (25.0%) | 20 (31.3%) | 9 | (26.5%) |

| | | | |
|---|---|---|---|
| SAEs related to Study Agent | 0 | 1 (1.6%) | 0 |

TABLE 9
Summary of Treatment Emergent Adverse
Events by SOC ≥10% (Safety Set)

| | Rivaroxaban | | | Aspirin | |
|---|---|---|---|---|---|
| | Part A | | Part B | Part B | |
| | N = 12 | | N = 64 | N = 34 | |
| Subject with 1 or more | 11 | (91.7%) | 55 (85.9%) | 29 | (85.3%) |
| TEAEs | | | | | |
| Infections and infestations | 8 | (66.7%) | 40 (62.5%) | 22 | (64.7%) |
| Respiratory, Thoracic, and | 5 | (41.7%) | 29 (45.3%) | 9 | (26.5%) |
| Mediastinal Disorders | | | | | |
| Gastrointestinal Disorders | 6 | (50.0%) | 19 (29.7%) | 9 | (26.5%) |
| Injury, Poisoning, and | 4 | (33.3%) | 18 (28.1%) | 10 | (29.4%) |
| Procedural Complications | | | | | |
| Sitin and Subcutaneous | 3 | (25.0%) | 19 (29.7%) | 9 | (26.5%) |
| Tissue Disorders | | | | | |
| General Disorders and | 1 | (8.3%) | 17 (26.6%) | 8 | (23.5%) |
| Administration Site | | | | | |
| Conditions | | | | | |
| Vascular Disorders | 2 | (16.7%) | 3 (4.7%) | 1 | (2.9%) |

TABLE 10
Summary of Adverse Events

| | Rivaroxaban | | | ASA | |
|---|---|---|---|---|---|
| | Part A | | Part B | Part B | |
| | N = 12 | | N = 64 | N = 34 | |
| Participants with 1 or more | 11 | (92%) | 55 (86%) | 29 | (85%) |
| Adverse Events | | | | | |
| Infections | 8 | (67%) | 40 (63%) | 22 | (65%) |
| Respiratory, Thoracic, and | 5 | (42%) | 29 (45%) | 9 | (26%) |
| Mediastinal Disorders | | | | | |
| Pleural Effusion | 3 | (25%) | 12 (19%) | 2 | (6%) |
| Gastrointestinal Disorders | 6 | (50%) | 19 (30%) | 9 | (26%) |
| Injury, Poisoning, and | 4 | (33%) | 18 (28%) | 10 | (29%) |
| Procedural Complications | | | | | |
| Skin and Subcutaneous | 3 | (25%) | 19 (30%) | 9 | (26%) |
| Tissue Disorders | | | | | |
| General Disorders and | 1 | (8%) | 17 (27%) | 8 | (24%) |
| Administration Site | | | | | |
| Conditions | | | | | |
| Vascular Disorders | 2 | (17%) | 3 (5%) | 1 | (3%) |
| Participants with 1 or | 6 | (50%) | 18 (28%) | 8 | (24%) |
| more Serious Adverse | | | | | |
| Events | | | | | |
| Infections | 3 | (25%) | 5 (8%) | 4 | (12%) |
| Respiratory, Thoracic, and | 2 | (17%) | 9 (14%) | 3 | (9%) |
| Mediastinal Disorders | | | | | |
| Pleural Effusion | 2 | (17%) | 9 (14%) | 2 | (6%) |

ASA = acetylsalicylic acid
Note:
All adverse events were Treatment-emergent. Treatment-emergent is defined as an adverse event or serious adverse event that occurs after the first dose and up to 2 days after the last dose of study drug.
Note:
Participants are counted only once for any given event, regardless of the number of times they experienced the event.
Note:
Percentages calculated with the number of participants in each group as denominator.
Note:
The organ classes are sorted in descending order of incidence >10% based on rivaroxaban

Pharmacokinetic and Pharmacodynamic

The main objective of Part A of the study was to confirm that subjects receiving the body weight-adjusted rivaroxaban dosing regimen used in the UNIVERSE study were able to achieve rivaroxaban exposures comparable to that observed in subjects who received the therapeutic dose of rivaroxaban 10 mg daily in adult studies (e.g., ODIXa-HIP II and KNEE studies). The body weight-adjusted rivaroxaban dosing table was generated by physiologically based pharmacokinetic (PBPK) modeling in children 2 to 8 years of age after the Fontan procedure. Once confirmed, the body weight-adjusted dosing regimen was used in Part B of the study.

The range of plasma rivaroxaban concentrations observed in the pediatric participants of this study validated the PBPK modeling and simulation predictions of the body weight-adjusted dosing regimen administered to pediatric post-Fontan patients as most of the plasma rivaroxaban concentrations in samples collected from the pediatric subjects on Day 4 and Month 3 in UNIVERSE study were within the range of concentrations reported in adults. No bleeding events were reported for the outliers.

The observed plasma concentration-time data of rivaroxaban of the full UNIVERSE data-set were adequately described by a 2-compartment population PK linear model. The model was parameterized in terms of CL/F estimated at 3.30 L/h, the Vc/F estimated at 17.6 L, Q/F estimated at 1.09 L/h, and Vp/F estimated at 33.4 L/h for a subject with body weight of 15 kg (median weight of the UNIVERSE study population). Rivaroxaban CL/F and Vc/F were scaled exponentially with body weight and the exponent was estimated to be 1.01 and 1.20, respectively. The first-order absorption rate constant was estimated to be 1.12/h. As in the EINSTEIN Jr population PK model, the dose-dependent relative bioavailability, F, was adequately described by the previously reported F function of adults after normalization of dose by weight.

The rivaroxaban concentrations observed in this study were superimposed onto the adult reference range of simulated concentrations from the study ODIXa-HIP-OD. The adult reference range was defined as the 2.5^{th} to 97.5^{th} percentile range of simulated concentrations based on the adult reference study ODIXa-HIP-OD, which enrolled adults who underwent a total hip replacement and were administered 10 mg QD dose of rivaroxaban.

The steady-state exposure metrics (AUC_{24h,ss}, C_{max,ss}, and C_{trough,ss}) of rivaroxaban in the UNIVERSE study are descriptively summarized in Table 4. The geometric mean AUC_{24h,ss}, the primary PK metric for exposure matching, was similar between patients in this study and the adult reference. Furthermore, the 90% Cls of the geometric means for these 2 groups largely overlapped. Since patients in the UNIVERSE study received a BID dosing regimen that was intended to match the 10 mg QD dosing in adults, the rivaroxaban concentrations in the pediatric patients had a narrower range with slightly lower Cmax,ss and slightly higher C_{trough,ss} in comparison to the adult reference ranges which was based on a QD regimen. Ratios of geometric mean exposures (AUC_{24h,ss}, C_{max,ss}, and C_{trough,ss}) for patients in the UNIVERSE study were compared with the adult reference (Table 4). The ratio for AUC_{24h,ss} was 0.96 and the corresponding 90% Cls were (0.87, 1.07). The BID dosing regimen in this study in comparison to the QD regimen in the adult reference, the geometric mean ratio for Cmax,ss was slightly lower than 1 and the geometric mean ratio for C_{trough,ss} was slightly higher than 1. These results demonstrated overall similarity in rivaroxaban exposures between this pediatric population and the adult reference.

Scatter plots were constructed to compare the exposure metrics (AUC_{24h,ss}, C_{max,ss}, and C_{trough,ss}) versus body weight between results from the UNIVERSE study and the adult reference at rivaroxaban 10 mg QD. The AUC_{24h,ss} from this study was largely contained within the 2.5^{th} to 97.5^{th} percentile range of the adult reference, indicating that the overall rivaroxaban exposures in patients in the UNIVERSE study were similar to those in adults. Patients in the UNIVERSE study who had relatively lower body weights tended to have slightly higher exposure as compared to the adult reference and predictions using the EINSTEIN Jr population PK model. However, the overall PK characteristics were similar between the UNIVERSE study and the EINSTEIN Jr program, with exposure metrics (AUC_{24h,ss}, C_{max,ss}, and C_{trough,ss}) largely overlapping with the pediatric prediction range based on the EINSTEIN Jr population PK model assuming the same dose regimen as the UNIVERSE study.

PK/PD Results from the UNIVERSE Study

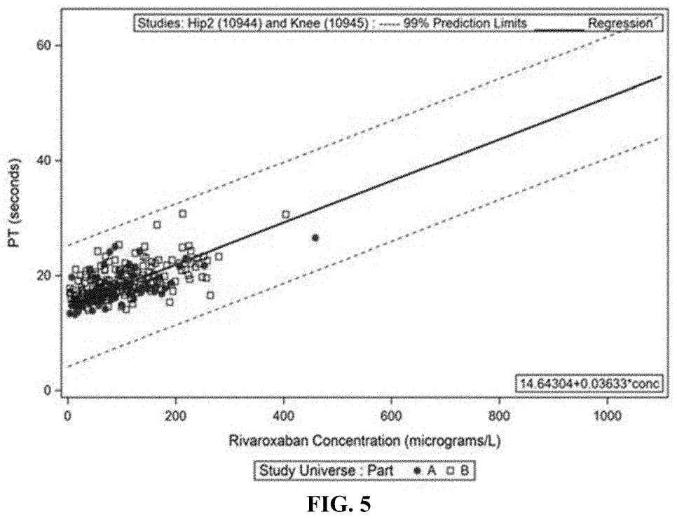

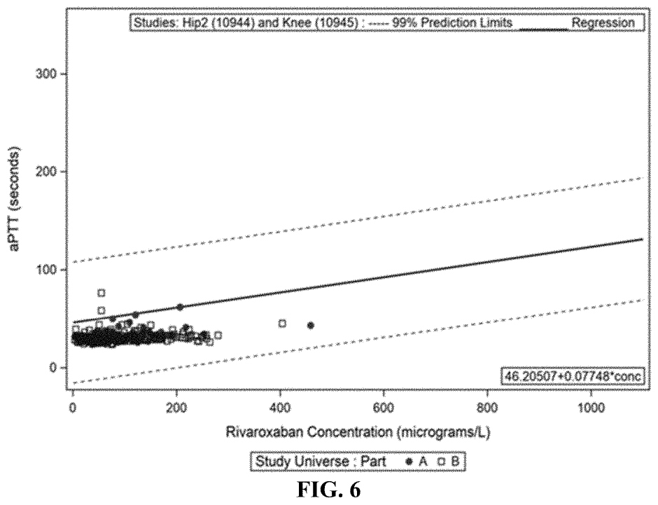

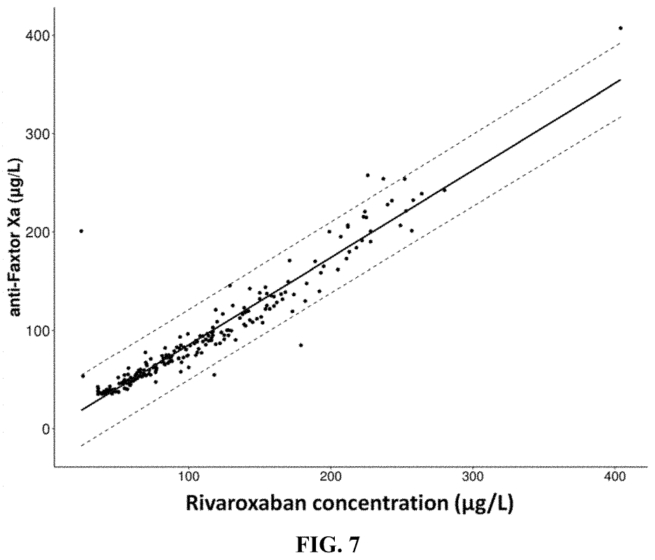

Since the adult PK reference study ODIXa-HIP-OD used an insensitive PT assay and could therefore not be used for reference, adult PD reference ranges were alternatively obtained from the adult Phase II hip and knee replacement studies ODIXa-HIP2 and ODIXa-KNEE, during which rivaroxaban was administered BID. As the PK/PD relationship is independent of the dosing frequency, it is appropriate to use these data for reference. Prothrombin time values as a function of rivaroxaban concentration were similar to the adult reference range as illustrated in FIG. 5. Activated thromboplastin time as a function of time is illustrated in FIG. 6. The slope for aPTT versus rivaroxaban concentration relationship was less steep in comparison to adult reference, which may be due to the low sensitivity of aPTT assays and its known large variability between studies due to sensitivity to reagents and experimental conditions. Anti-factor Xa activity correlated strongly (R-squared=0.901) with rivaroxaban concentrations in UNIVERSE (FIG. 7).

Exposure-Efficacy and Exposure-Safety Results from the UNIVERSE Study

Within the rivaroxaban treatment group, 2 thrombotic events, including 1 venous thrombosis and 1 pulmonary embolism, were observed in 2 patients, respectively. Bleeding events were observed in 27 patients. Three patients experienced multiple bleeding events and 24 patients experienced 1 event. Among the bleeding events, 1 major bleeding, 5 clinically relevant non-major bleeding, and 24 trivial bleeding events were observed.

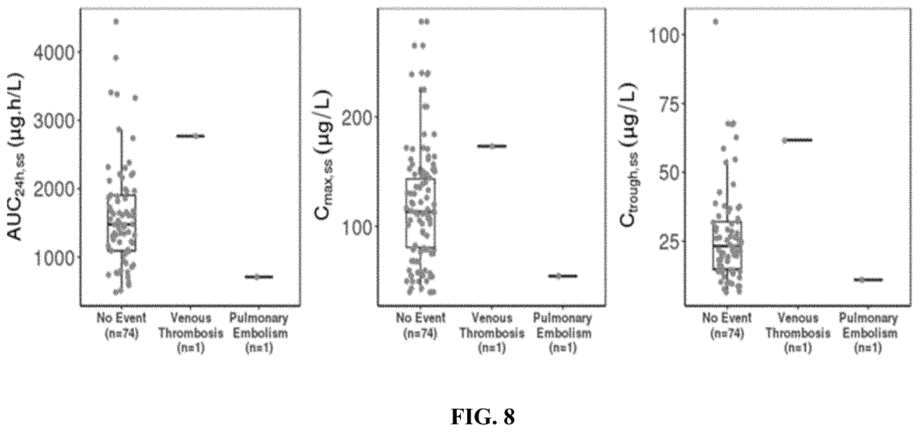

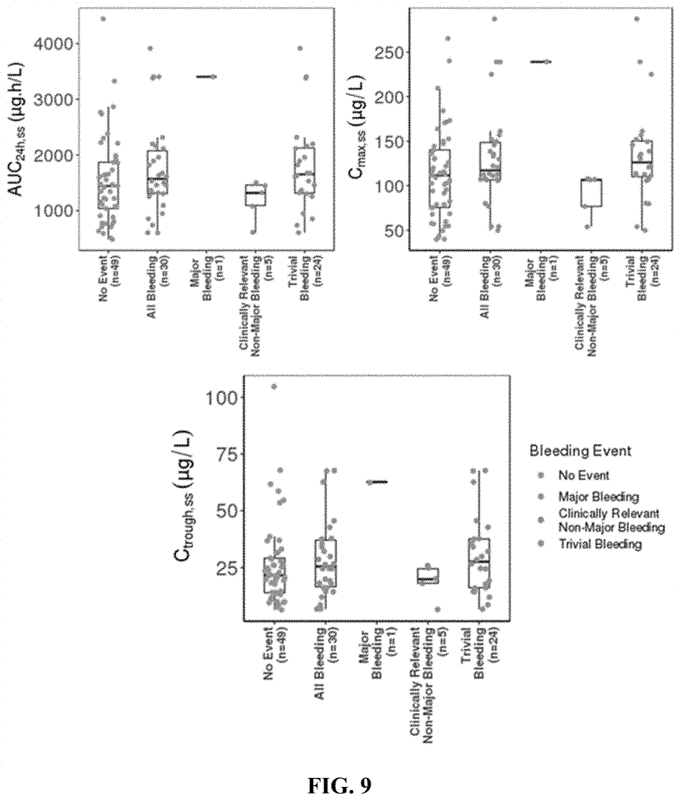

Exposure-response relationships in patients treated with rivaroxaban were visually investigated by comparing the exposure metrics (AUC_{24h,ss}, C_{max,ss}, and C_{trough,ss}) in patients with or without thrombotic or bleeding events, and with or without bleeding events (FIGS. 8 and 9). The ranges of AUC_{24h,ss}, C_{max,ss}, and C_{trough,ss} largely overlapped between patients with or without thrombosis or bleeding events. These results suggest that, within the exposure range observed, there was no apparent exposure-response relationship between rivaroxaban exposure and thrombosis or bleeding events.

Benefit-Risk Assessment

Benefit-risk balance was assessed using excess number of events defined as the absolute proportion difference between the Part B rivaroxaban and aspirin groups scaled to 1,000 subjects to reflect benefits and risks on a population level.

For 1,000 subjects treated with rivaroxaban, rivaroxaban would be expected to prevent 73 thrombotic events (primary efficacy outcome) compared with aspirin. Rivaroxaban would be expected to cause 16 more events of ISTH major bleeding, but 25 less events of clinically relevant non-major bleeding than with aspirin (Table 11).

The benefit-risk profile of rivaroxaban in children post-Fontan procedure appears favorable compared with aspirin.

TABLE 11
Excess Number of Key Efficacy and Safety Outcomes Per 1000 Subjects,
Up-to-End-of Treatment; Full Analysis Set (Study 39039039CHD3001)

| | | | | | Part B: | |
|---|---|---|---|---|---|---|
| | | | | | Rivaroxaban-Aspirin | |
| | | | | | Excess | |
| | | | | | number of | |
| | Rivaroxaban part B | | Aspirin part B | | events per | |
| | No.(%) of | | No.(%) of | | 1,000 | |
| | subjects with | | subjects with | | subjects | NNT or |
| | event | 95% CI | event | 95% CI | (95% CI) | NNH |
| Analysis set: Full | 64 | | 34 | | | |
| Efficacy | | | | | | |
| Any thrombotic event (primary | 1/64 | (NA, NA) | 3/34 | (0.0%-19.8%) | −72.6 | −14 |
| efficacy outcome) | (1.6%) | | (8.8%) | | | |
| Venous thromboembolism | 1/64 | (NA, NA) | 2/34 | (0.0%-15.3%) | −43.2 | −24 |
| | (1.6%) | | (5.9%) | | | |
| Venous thrombosis | 0 | (NA, NA) | 2/34 | (0.0%-15.3%) | −58.8 | −17 |
| | | | (5.9%) | | | |
| Pulmonary embolism | 1/64 | (NA, NA) | 0 | (NA, NA) | 15.6 | 64 |
| | (1.6%) | | | | | |
| Arterial/intracardiac thrombosis | 0 | (NA, NA) | 0 | (NA, NA) | 0.0 | NA |
| Ischemic stroke | 0 | (NA, NA) | 1/34 | (NA, NA) | −29.4 | −34 |
| | | | (2.9%) | | | |
| All-cause mortality | 0 | (NA, NA) | 0 | (NA, NA) | 0.0 | NA |
| Safety | | | | | | |
| Major bleeding (primary safety | 1/64 | (NA, NA) | 0 | (NA, NA) | 15.6 | 64 |
| outcome) | (1.6%) | | | | | |
| Fatal or critical site bleeding | 0 | (NA, NA) | 0 | (NA, NA) | 0.0 | NA |
| Fall in hemoglobin ≥2 g/dL or | 1/64 | (NA, NA) | 0 | (NA, NA) | 15.6 | 64 |
| transfusion of the equivalent ≥2 | (1.6%) | | | | | |
| units of packed red blood cell | | | | | | |
| or whole blood in adults | | | | | | |
| Clinically relevant non-major | 4/64 | (0.0%-13.0%) | 3/34 | (0.0%-19.8%) | −25.7 | −39 |
| (CRNM) bleeding | (6.3%) | | (8.8%) | | (−160.5, 109.1) | |
| Major and clinically relevant non- | 5/64 | (0.5%-15.2%) | 3/34 | (0.0%-19.8%) | −10.1 | −99 |
| major bleeding | (7.8%) | | (8.8%) | | (−148.4, 128.2) | |
| Trivial (minimal) bleeding | 21/64 | (20.5%-45.1%) | 12/34 | (17.8%-52.8%) | −24.8 | −41 |
| | (32.8%) | | (35.3%) | | (−244.9, 195.3) | |
| SAEs | 18/64 | (16.3%-39.9%) | 8/34 | (7.8%-39.3%) | 46.0 | 21 |
| | (28.1%) | | (23.5%) | | (−156.7, 248.7) | |

Note:
CIAC = Central Independent Adjudication Committee; CI = Confidence Interval; NNT = number needed to treat to benefit; NNH = number needed to treat to harm.
Note:
Venous thromboembolism, arterial/intracardiac thrombosis, ischemic stroke, death, major bleeding (not including its subcategories), CRNM bleeding, and trivial bleeding summaries are based on CIAC adjudication. SAEs and major bleeding subcategories, are based on investigator report.
Note:
Full Analysis Set: all subjects in Part A who receive at least 1 dose of study agent and all subjects in Part B who are randomized and receive at least 1 dose of study agent.
Note:
Up-to-End-of Treatment is defined as the period starting from first dose of study agent to end of treatment visit.
Note:
Percentages calculated with the number of subjects in each group as denominator.
Note:
Confidence intervals presented are based on the normal approximation. No CI is provided if the number of events is 0 or 1 in either group.
Note:
NNT/NNH calculated as the reciprocal of the corresponding risk difference. Values for efficacy endpoints are NNTs.
Values for safety endpoints are NNHs. For both NNT and NNH, positive values favor Aspirin and negative values favor Rivaroxaban.

### Discussion

Part B of the study compared a body weight-adjusted dosing regimen of rivaroxaban (dosed to match the exposure of the therapeutic dose of 10 mg daily in adults) with aspirin (2:1 randomization) in children aged 2 to 8 years with congenital heart disease post-Fontan procedure. Baseline characteristics were well balanced between treatment groups. Most of the subjects (95.5%) completed study participation. Median duration of study exposure during the intended 12-month treatment duration was 359 days amongst all treatment groups. The study met FDA Written Request's exposure requirement: “at least 50 subjects should be exposed to rivaroxaban for 300 days” since 69 pediatric subjects in the study were exposed to rivaroxaban for 300 days. Pediatric subjects in Part B of the study treated with rivaroxaban experienced fewer thromboembolic events than those treated with aspirin (rivaroxaban 1 [1.6%] vs aspirin 3 [8.8%]). There was 1 major bleeding event (epistaxis, non-critical site, blood transfusion) in the rivaroxaban Part B group and none in the aspirin group. Non-major clinically relevant bleeding in the rivaroxaban group vs aspirin was rivaroxaban 4 [6.6%] vs aspirin 3 [8.8%]. There were no new safety signals uncovered for rivaroxaban. In Part A, PK results showed that rivaroxaban exposure in the study matched the rivaroxaban exposure reached in adults in the hip and knee replacement VTE prevention studies taking the therapeutic 10 mg total daily dose. The benefit-risk profile of rivaroxaban in children post-Fontan procedure appears favorable compared with aspirin.

### Model-Informed Dose-Exposure Extrapolation

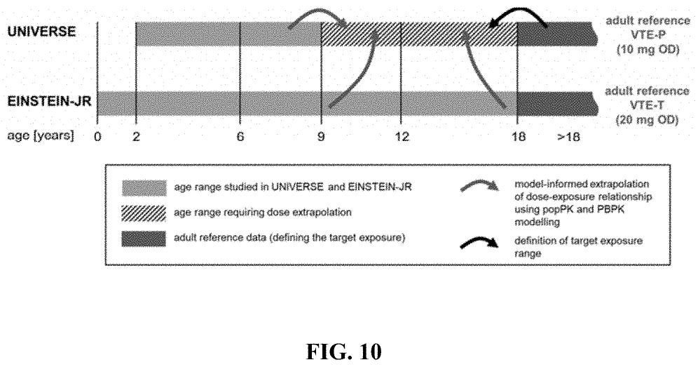

To extrapolate the dose-exposure relationship of post-Fontan patients in the age range of 2 to 8 years to post-Fontan patients aged to 18 years, a model-informed bridging approach using physiologically-based pharmacokinetic (PBPK) as well as population pharmacokinetic (popPK) approaches were applied that integrated rivaroxaban PK data from the EINSTEIN-JR program and the UNIVERSE study and also considered the rivaroxaban dose-exposure relationship in adult patients who were enrolled in VTE prevention studies. The available clinical database and the concept for the bridging approach are outlined in FIG. 10. In the UNIVERSE study, the dose-exposure relationship of rivaroxaban was established in post-Fontan subjects from 2 to 8 years of age. From the EINSTEIN-JR program, the dose-exposure relationship was known for pediatric VTE patients. The EINSTEIN-JR database includes phase I-III data of the EINSTEIN-JR program as well as the Part A PK data from the UNIVERSE study over the whole age range of 0-18 years and a body weight range from 2.7 to 194 kg. From a total of 524 patients in the EINSTEIN-JR PK/PD database, 262 (50.0%) were in the age range between 9 and 18 years.

In total, the pediatric rivaroxaban PK database used for this extrapolation task consisted of 76 post-Fontan patients in the age range from 2 to 8 years (12 from Part A and 64 from Part B of the UNIVERSE study) and 512 patients from birth to <18 years studied in the EINSTEIN-JR program. 262 EINSTEIN-JR patients were in the age range from ≥9 to 18 years, i. e. the age range for which the dose-exposure relationship is to be extrapolated in post-Fontan patients. The target range of rivaroxaban exposure for post-Fontan patients ≥9 to 18 years of age was the same as for post-Fontan patients who are 2 to 8 years of age (ie., exposure that was achieved with 10 mg once daily in adults in VTE-prevention [VTE-P] studies).

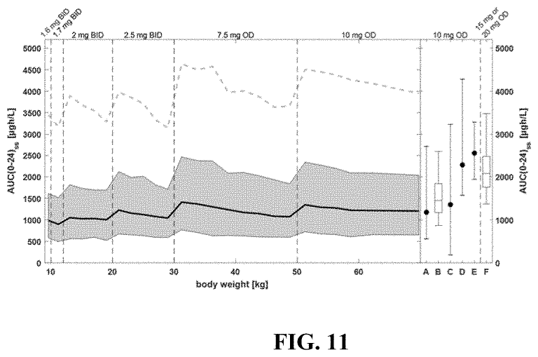

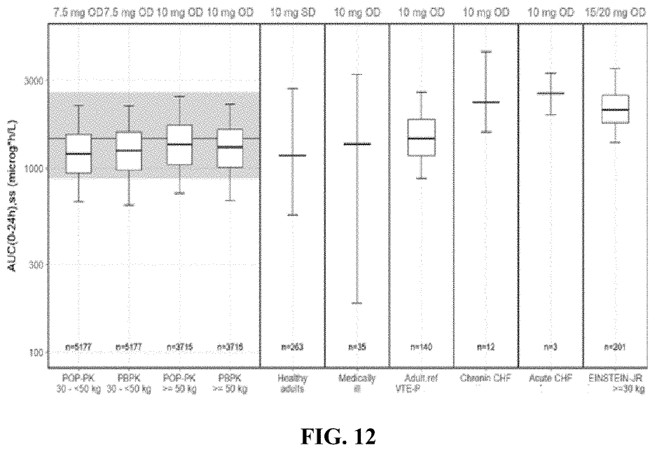

In a first model evaluation and qualification step, the adequacy of the Fontan-PBPK model and the EINSTEIN-JR popPK model to describe the rivaroxaban PK data observed in the UNIVERSE study in post-Fontan patients 2 to 8 years of age was assessed. The Fontan-PBPK model and EINSTEIN-JR popPK model were considered to be adequate and qualified for predicting the dose-exposure relationship of pediatric post-Fontan subjects ≥30 kg, because the models described rivaroxaban exposure of pediatric post-Fontan subjects ≥5 years of age very well. Deviations between model-predicted and observed data were seen mostly in subjects <5 years of age. No adjustments were made to any of the parameters of the initial Fontan PBPK model in order to predict the dose-exposure relationship of post-Fontan subjects ≥9 years and ≥30 kg. The PBPK and popPK models were then used to provide population estimates for rivaroxaban exposure for proposed thromboprophylactic doses in pediatric post-Fontan subjects ≥30 kg. FIG. 11 shows the rivaroxaban AUC(0-24)ₛₛ predicted by the Fontan-PBPK model after extension to pediatric post-Fontan patients aged ≥9 to 18 years. Under the assumption that hepatic function is not impaired in the post-Fontan patients, the PBPK simulations support that 7.5 mg rivaroxaban once daily (BW from 30 to <50 kg) and 10 mg once daily (BW ≥50 kg) are suitable doses to yield exposure that is similar to exposure observed in adult VTE-P patients receiving 10 mg once daily. Under the assumption of severe hepatic impairment, rivaroxaban exposure is expected to increase as indicated by the dotted line in FIG. 11. The range of exposure predicted under this assumption largely overlaps with the exposure observed in adult CHF subjects receiving 10 mg rivaroxaban once daily (shown as references D and E in FIG. 11) and partially overlaps with the exposure observed in EINSTEIN-JR subjects with body weight ≥30 kg receiving either 15 mg once daily (body weight of 30 to <50 kg) or 20 mg once daily (body weight 50 kg, shown as reference F in FIG. 11. FIG. 12 summarizes the rivaroxaban dose-exposure predictions for post-Fontan patients 30 kg by the PBPK and popPK modelling approaches in comparison to adult and EINSTEIN-JR reference data. The two different modelling approaches come to very similar results and consistently support the suitability of the proposed dosing regimen (Table 12) under the assumption that liver function is not severely impaired in the post-Fontan subjects. The proposed doses for thromboprophylaxis in pediatric post-Fontan patients weighing 30 kg and above are one-half of the rivaroxaban doses that were established in the EINSTEIN-JR program for the treatment of VTE in pediatric subjects for the corresponding body weight ranges.

TABLE 12
Rivaroxaban dose regimens established for pediatric subjects
in the EINSTEIN-JR study and proposed dose regimen for
thromboprophylaxis in pediatric post-Fontan subjects

| | | doses for pediatric |
|---|---|---|
| | EINSTEIN-JR doses | post-Fontan patients |
| | (matching 20 mg once | (targeting 10 mg once |
| Body weight | daily in adults) | daily in adults) |
| 30 kg to <50 kg | 15 mg once daily | 7.5 mg once daily |
| ≥50 kg | 20 mg once daily^{a} | 10 mg once daily |

^{a}15 mg in Japan

Based on the information from this study, methods for thromboprophylaxis are provided for children from ages 2 to 18, wherein there is a body weight-adjusted dosing regimen of rivaroxaban.

What is claimed is:

1. A method of thromboprophylaxis in a patient post-Fontan procedure, the method comprising administering to said patient rivaroxaban in an amount clinically proven safe and clinically proven effective for thromboprophylaxis in pediatric patients 2 years and older with congenital heart disease after the Fontan procedure

2. The method of claim 1, wherein said patient is an age of 2 to 8 years.

3. The method of claim 1, wherein the administering is once or twice daily.

4. The method of claim 1, wherein the rivaroxaban is in an oral suspension or a tablet.

5. The method of claim 4, wherein 1.1 mg of rivaroxaban per dosage is administered for said patient weighing from 7 kg to less than 8 kg.

6. The method of claim 4, wherein 1.6 mg of rivaroxaban per dosage is administered for said patient weighing from 8 kg to less than 10 kg.

7. The method of claim 4, wherein 1.7 mg of rivaroxaban per dosage is administered for said patient weighing from 10 kg to less than 12 kg.

8. The method of claim 4, wherein 2.0 mg of rivaroxaban per dosage is administered for said patient weighing from 12 kg to less than 20 kg.

9. The method of claim 4, wherein 2.5 mg of rivaroxaban per dosage is administered for said patient weighing from 20 kg to less than 30 kg.

10. The method of claim 4, wherein 7.5 mg of rivaroxaban per dosage is administered for said patient weighing from 30 kg to less than 50 kg.

11. The method of claim 4, wherein 10 mg of rivaroxaban per dosage is administered for said patient weighing 50 kg or more.

12. The method of claim 1, wherein the administering is for a duration up to 12 months.

13. The method of claim 1, wherein the administering results in a steady-state exposure of rivaroxaban at a geometric mean exposure AUC_{ss,24h} of 70% to 143% of AUC_{ss,24h} 1494 μg*h/L.

14. The method of claim 1, wherein the administering results in a steady-state exposure of rivaroxaban at a geometric mean exposure AUC_{ss,24h} that is within the range of 1317 to 1576 μg*h/L.

15. The method of claim 1, wherein rivaroxaban is initially administered to the patient within 4 months, such as within 1, 2, 3, 4, 5, 6, 7, 8, 9, 10, 11, 12, 13, 14, 15 or 16 weeks, after the Fontan procedure is completed.

16. The method of claim 1, wherein the patient is a responder to said treatment as measured by the absence of the incidence of any thrombotic event in the patient for a period of 12 months or more from first administration.

17. The method of claim 16, wherein said patient is a patient having an extracardiac conduit.

18. A method of thromboprophylaxis in a pediatric patient after a Fontan procedure, the method comprising orally administering to said patient once daily or twice daily rivaroxaban in an amount clinically proven safe and clinically proven effective for thromboprophylaxis, wherein the administering results in a steady-state exposure of rivaroxaban at a geometric mean exposure AUC_{ss,24h} that is within the range of 1317 to 1576 μg*h/L and the patient is a responder to said treatment as measured by the absence of the incidence of any thrombotic event in the patient for a period of 12 months or more from first administration.

19. The method of claim 18, wherein the rivaroxaban is in an oral suspension or a tablet.

20. The method of claim 19, wherein the patient is a patient of from 2 years to 8 years old.
